(19) United States
(12) Patent Application Publication
(10) Pub. No.: US 2009/0042848 A1
(43) Pub. Date: Feb. 12, 2009

(54) COPPER COMPLEXES

(76) Inventors: Peter Lay, Newton (AU); Trevor Hambley, Ashbury (AU); Brenden Kennedy, Newtown (AU); Ying Morgan, Maroubra (AU)

Correspondence Address: FULBRIGHT & JAWORSKI, LLP, 1301 MCKINNEY, SUITE 5100, HOUSTON, TX, 77010-3095, US

(21) Appl. No.: 11/909,528

(22) PCT Filed: Mar. 24, 2006
(86) PCT No.: PCT/AU2006/000402

§ 371 Date: Jun. 11, 2008

Related U.S. Application Data
(60) Provisional application No. 60/664,867, filed on Mar. 24, 2005.

(30) Foreign Application Priority Data
Mar. 24, 2005 (AU) 2005901464
Mar. 30, 2005 (AU) PCT/AU2005/000442
Oct. 4, 2005 (AU) 2005905476
Oct. 5, 2005 (AU) 2005905479

(51) Int. Cl. A61K 31/555; C07F 15/00; A61P 29/00

(52) U.S. Cl. 514/184; 546/2; 548/402; 548/101; 514/188

(57) ABSTRACT

There are described mononuclear copper complexes having anti-inflammatory activity. The complexes include copper complexes of indomethacin. There are also provided methods for the prophylaxis or treatment of inflammation comprising administering such complexes to a mammalian subject.

This Application is the 35 USC § 371 Application of International Application No. PCT/AU2006/000402 filed Mar. 24, 2006, which claims priority to Patent Application in Australia No. 2005901464, filed Mar. 24, 2005; U.S. Patent Application Ser. No. 60/664,867, filed Mar. 24, 2005; International Application No. PCT/AU2005/000442, filed Mar. 30, 2005; Patent Application in Australia No. 2005905476, filed Oct. 4, 2005; and No. 2005905479, filed Oct. 5, 2005, all of which applications are incorporated by reference herein in their entirety.

### FIELD OF THE INVENTION

The present invention relates to copper complexes containing ligands having anti-inflammatory activity, including copper complexes of indomethacin. The invention also relates to the use of the complexes in the treatment of inflammatory conditions in humans and animals.

### BACKGROUND

Non-steroidal anti-inflammatory drugs (NSAIDs) are used to treat a variety of inflammatory conditions in humans and animals. NSAIDs are, for example, used to treat inflammatory conditions such as rheumatoid arthritis, osteoarthritis, acute musculoskeletal disorders (such as tendonitis, sprains and strains), lower back pain (commonly referred to as lumbago), and inflammation, pain and edema following surgical or non-surgical procedures. However, many NSAIDs cause adverse effects in humans and animals, particularly adverse gastrointestinal effects.

Indomethacin is a NSAID and is effective in treating inflammatory conditions in humans and animals. However, indomethacin can cause severe adverse gastrointestinal effects in humans and animals, particularly when administered orally. In humans, oral administration of indomethacin can cause ulcerations in the esophagus, stomach, duodenum and intestines, and some fatalities have been reported. In dogs, indomethacin causes fatal gastrointestinal haemorrhaging. Adverse effects associated with the topical administration of indomethacin have been reported in “Anti-inflammatory activity of Indomethacin following topical application”, Amico-Roxas, M.; Matera, M.; Caruso, A.; Puglisi, G.; Bernardini, R.; Rinaldo, G. Rivista Europea per le Scienze Mediche e Farmacologiche (1982), 4(2), 199-204. Adverse gastrointestinal effects have also been reported for administration of indomethacin by suppository. The adverse effects of indomethacin have limited the use of indomethacin in the treatment of inflammatory conditions in humans and animals.

Indomethacin has the structure:

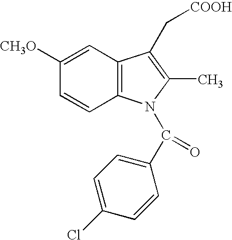

In indomethacin, the benzene ring has a chloro substituent at the 3-position. Similar compounds in which the benzene ring is substituted at the 3-position with a halo substituent other than Cl, the benzene ring is substituted with a halo substituent at a position other than the 3-position, and/or the benzene ring has two or more halo substituents, also have similar anti-inflammatory activity to indomethacin (Loll, P. J.; Picot, D.; Ekabo, O.; Garavito, R. M. Biochemistry 1996, 35, 7330-7340; Touhey, S.; O'Connor, R.; Plunkett, S.; Maguire, A.; Clynes, M. Eur. J. Cancer 2002, 38, 1661-1670; Fukaya, C.; Naito, Y.; Hanada, S.; Watanabe, M.; Yokoyama, K. Preparation of fluorinated indoleacetic acid derivatives as antiinflammatory drugs. U.S. (1989), 6 pp. Cont.-in-part of U.S. Ser. No. 788,445, abandoned). Some of these compounds show selectivity for inhibition of the COX-II enzyme relative to the COX-I enzyme, and cause less gastrointestinal toxicity than indomethacin (Weder, J. E.; Dillon, C. T.; Hambley, T. W.; Kennedy, B. J.; Lay, P. A.; Biffin, J. R.; Regtop, H. L.; Davies, N. M. Coord. Chem. Rev. 2002, 232, 95-126). Other compounds having a similar structure to indomethacin and having anti-inflammatory activity are described in WO2005/002525.

It has been found that dinuclear metal complexes of indomethacin (containing two metal coordination centres) cause less adverse side effects, and result in increased uptake of the drug, compared to free indomethacin. For example, the oral administration of the dinuclear Cu(II) complex of indomethacin, bis(N,N-dimethylformamide)tetrakis-μ-(O,O′-Indo)dicopper(II) complex ([Cu₂(Indo)₄(DMF)₂]), has been found to cause less gastrointestinal toxicity than indomethacin; and it has been claimed that the complex has increased anti-inflammatory activity compared to indomethacin. The mechanism of the reduced gastrointestinal toxicity has not been elucidated. However, it is believed that it is at least in part due to the complex being more lipophilic than indomethacin, which leads to greater absorption of the complex.

Compositions containing this complex, sold under the name Cu-Algesic, have been used in veterinary practice in Australia, New Zealand, South Africa and other countries. These compositions are in the form of a tablet or a paste.

All the metal complexes of indomethacin described to date as having reduced gastrointestinal toxicity compared to indomethacin are dinuclear metal complexes. Recently the first mononuclear indomethacin complex, [Cu(Indo)₂(Py)₃], was prepared and a preliminary X-ray structure was reported in which both of the indomethacin ligands were monodentate (Preparation and Characterization of Dinuclear Copper-Indomethacin Anti-Inflammatory Drugs. Morgan, Y. R.; Turner, P.; Kennedy, B. J.; Hambley, T. W.; Lay, P. A.; Biffin, J. R.; Regtop, H. L; Warwick, B. Inorg. Chim. Acta 2001, 324, 150-161). While no information on gastrointestinal toxicity has been reported for this mononuclear complex, mononuclear Zn(II)-Indo complexes have been found to have greater gastrointestinal toxicity than the Zn(II)-Indo dimers (Zhou, Q., PhD Thesis, University of Sydney, 2001).

While mononuclear complexes with other carboxylate NSAIDs have been reported (eg. see Copper Complexes of Non-steroidal Anti-inflammatory Drugs: An Opportunity yet to be Realized Weder, J. E.; Dillon, C. T.; Hambley, T. W.; Kennedy, B. J.; Lay, P. A.; Biffin, J. R.; Regtop, H. L.; Davies, N. M. Coord. Chem. Rev. 2002, 232, 95-126; Copper and Zinc Complexes as Anti-Inflammatory Drugs. Dillon, C. T.; Hambley, T. W.; Kennedy, B. J.; Lay, P. A.; Weder, J. E.; Zhou, Q. in “Metal Ions and Their Complexes in Medication”, Vol. 41 of ‘Metal Ions in Biological Systems’; Sigel, A.; Sigel, H., Eds.; M. Dekker, Inc., New York & Basel, 2004, Ch. 8, pp 253-277), it is the understanding of the present inventors no mononuclear metal complexes of indomethacin other than [Cu(Indo)₂(Py)₃] have been reported despite the dinuclear complexes of the NSAID having been known for over a decade.

### SUMMARY OF THE INVENTION

In a first aspect, the present invention provides a complex of the formula (1):

[Cu(η²-L¹)₂L₂]^{p} (1)

wherein “η²-L¹” is a bidentate ligand of the formula L¹:

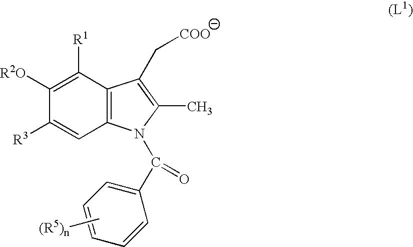

wherein:

R¹ is H or halo (i.e., Cl, F, Br or I);

R² is H; a C₁ to C₆ alkyl, an alkenyl or an alkynyl, where the C₁ to C₆ alkyl, alkenyl or alkynyl may be optionally substituted; or

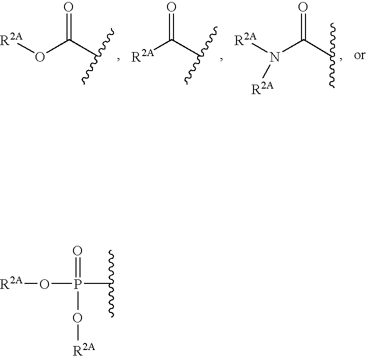

wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;

R³ is H or halo;

each R⁵ is independently selected from the group consisting of halo, —CH₃, —CN, —OCH₃, —SCH₃ and —CH₂CH₃, where the —CH₃, —OCH₃—SCH₃ or —CH₂CH₃ may be optionally substituted;

n is 1, 2, 3, 4 or 5;

each L is independently selected and is a monodentate ligand; and

p is the charge of the complex.

When R² is a C₁ to C₆ alkyl, an alkenyl or an alkynyl, the C₁ to C₆ alkyl, alkenyl or alkynyl may be substituted with one or more substituents. The one or more substituents may, for example, be independently selected from the group consisting of halo, —OH, —COOH and —NH₂.

When R^{2A} is a C₁ to C₆ alkyl, an alkenyl, an alkynyl, an aryl, a cycloalkyl or an arylalkyl, the C₁ to C₆ alkyl, alkenyl, alkynyl, aryl, cycloalkyl or arylalkyl may be substituted with one or more substituents. The one or more substituents may, for example, be independently selected from the group consisting of halo, —OH, —COOH and —NH₂.

When R⁵ is —CH₃, —OCH₃—SCH₃ or —CH₂CH₃, the —CH₃, —OCH₃, —SCH₃ or —CH₂CH₃ may be substituted with one or more substituents. The one or more substituents may, for example, be independently selected from the group consisting of halo, —OH, —COOH and —NH₂.

R¹ is typically H.

R³ is typically H.

R² is typically CH₃

Each R⁵ is typically halo (i.e. F, Cl, Br or I), and n is typically 1, 2 or 3.

L¹ may for example be Indo.

The present inventors have surprisingly found that one or more embodiments of complexes of formula (1) cause less adverse gastrointestinal effects (particularly less adverse effects in the small intestines) than an equimolar dose of the group of the formula L¹ in the form of the free compound L¹H (where L¹ is as defined above). The present inventors have also found that one or more embodiments of complexes of formula (1) cause less than, or similar adverse gastrointestinal effects to, an equimolar dose of L¹ in the form of a dinuclear copper complex containing the ligand L¹ as a bridging ligand. The lower or similar gastrointestinal toxicity of the mononuclear complexes of formula (1) compared to dinuclear complexes is different to what was observed for zinc-indomethacin complexes (Dillon, C. T.; Hambley, T. W.; Kennedy, B. J.; Lay, P. A.; Zhou, Q.; Davies, N. M.; Biffin, J. R.; Regtop, H. L. Chem. Res. Toxicol., 2003, 16, 28-37). The present inventors have further found that complexes of formula (1) cause surprisingly less adverse gastrointestinal effects (particularly less adverse effects in the small intestines) than an equimolar dose of L¹ in the form of a mononuclear copper-indomethacin complex containing one or more monodentate ligands of the formula L¹.

In a second aspect, the present invention provides a pharmaceutical composition comprising a complex according to the first aspect of the present invention and a pharmaceutically acceptable carrier. The composition may be suitable for administration by oral administration, topical application, as a suppository, by inhalation or by some other route.

In a third aspect, the present invention provides a method of treating an inflammatory condition in a human or animal, the method comprising administrating to the human or animal a therapeutically effective amount of a complex according to the first aspect of the present invention. The animal may, for example, be a dog, a cat, a cow, a horse, a camel, etc. The complex may be administered orally, topically, by injection, by suppository, by inhalation or by some other route.

In a fourth aspect, the present invention provides the use of a complex of formula (1) in the manufacture of a medicament for the treatment of an inflammatory condition.

All publications mentioned in this specification are herein incorporated by reference. Any discussion of documents, acts, materials, devices, articles or the like which has been included in this specification is solely for the purpose of providing a context for the present invention. It is not to be taken as an admission that any or all of these matters form part of the prior art base or were common general knowledge in the field relevant to the present invention as it existed anywhere before the priority date of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The features and advantages of methods of the present invention will become further apparent from the following detailed description of preferred embodiments.

### BRIEF DESCRIPTION OF THE ACCOMPANYING DRAWINGS

FIG. 1 shows the UV-V is solution spectra of: (a) [Cu₂(Indo)₄(DMA)₂] (0.113 and 1.133 mg/mL in DMA); (b) [Cu(Indo)₂(Pyrro)₂] (0.1062 and 1.062 mg/mL in pyrrolidine); (c) [Cu₂(Indo)₄(THF)₂] (0.106 and 1.016 mg/mL in THF); (d) [Cu₂(Indo)₄(ACN)₂] (0.01036 and 1.036 mg/mL in ACN); (e) [Cu(Indo)₂(Py)₃] (0.1045 and 1.045 mg/mL in Py) and (f) IndoH (0.0132 mg/mL in DMF). The loss of intensity of the absorbance in the UV region in some solvents was due to the absorbance of the solvent.

FIG. 2 shows the IR spectra of: (a) [Cu(Indo)₂(Py)₃]; (b) [Cu(Indo)₂(Pyrro)₂]; (c) [Cu₂(Indo)₄(ACN)₂]; (d) [Cu₂(Indo)₄(THF)₂]; (e) [Cu₂(Indo)₄(DMA)₂]; (f) [Cu₂(OAc)₄(OH₂)₂]; and (g) IndoH in a KBr matrix.

FIG. 3 shows the X-band EPR spectra at room-temperature of (a) [Cu(Indo)₂(Py)₃] in pyridine solution; and of powders of (b) [Cu(Indo)₂(Py)₃]; (c) [Cu(Indo)₂(Pyrro)₂]; (d) [Cu₂(Indo)₄(ACN)₂]; (e) [Cu₂(Indo)₄(THF)₂]; and (f) [Cu₂(Indo)₄(DMA)₂].

FIG. 4 shows the X-ray powder diffraction patterns of (a) dinuclear [Cu₂(Indo)₄L₂] (L=DMA, THF or ACN); and (b) mononuclear [Cu(Indo)₂(Py)₃] complexes. The short vertical marks show the positions of the Bragg reflections expected from the results of all the single-crystal analyses. There is no reflection for [Cu₂(Indo)₄(ACN)₂].

FIG. 5 shows a series of graphs of the bond distances and Cu displacement in dinuclear complexes of the formula [Cu₂(Indo)₄L₂] (L=THF, DMF, DMA, DMSO or Py).

FIG. 6 shows the ORTEP³⁹ depiction of the mononuclear complex [Cu(Indo)₂(Py)₃] with atomic displacement parameters at the 20% level (150 K).

FIG. 7 shows the ORTEP³⁹ depiction of the mononuclear complex [Cu(Indo)₂(Pyrro)₂] with atomic displacement parameters at the 20% level.

FIG. 8 shows two graphs of the macroscopic gastrointestinal ulcerations observed in rats following oral administration with: (a) 2% (w/v) CMC solution (control); (b) IndoH (10 mg/kg); (c) Cu-acetate; and equimolar Indo and Cu doses of (d) physical mixture of Cu-acetate & IndoH; (e) [Cu₂(Indo)₄(DMF)₂]; (f) [Cu(Indo)₂(Py)₃]; and (g) [Cu(Indo)₂(Pyrro)₂] in CMC solution in (1) the stomach and (2) the small intestine. Each bar represents the mean±SEM for 4-18 rats.

FIG. 9 shows a graph of the effect on carrageenan-induced paw edema of oral administrated: (a) 2% (w/v) CMC solution (control); (b) IndoH (10 mg/kg); (c) Cu-acetate; and equimolar Indo and Cu doses of: (d) physical mixture of Cu-acetate & IndoH; (e) [Cu₂(Indo)₄(DMF)₂]; (f) [Cu(Indo)₂(Py)₃]; and (g) [Cu(Indo)₂(Pyrro)₂]; in 2% (w/v) CMC solution. Each bar represents the mean±SEM for 3-11 rats.

FIG. 10 shows the ORTEP³⁹ depiction of the mononuclear complex [Cu(Indo)₂(Im)₂] with atomic displacement parameters at the 20% level.

FIG. 11 shows the ORTEP³⁹ depiction of the mononuclear complex [Cu(Indo)₂(4-pic)₂] with atomic displacement parameters at the 20% level.

### DETAILED DESCRIPTION OF PREFERRED EMBODIMENTS OF THE INVENTION

In this specification, the abbreviation “IndoH” refers to the uncharged form of indomethacin, “Indo” refers to the deprotonated anionic form, “ACN” refers to acetonitrile, “THF” refers to tetrahydrofuran, “Py” refers to pyridine, “Pyrro” refers to pyrrolidine, “DMA” refers to N,N-dimethylacetamide, “DMSO” refers to dimethylsulfoxide, and “DMF” refers to N,N-dimethylformamide.

In this specification, the term “halo” refers to fluoro, chloro, bromo or iodo.

In this specification, the term “alkyl” used either alone or in a compound word such as “arylalkyl”, refers to a straight chain, branched or mono- or poly-cyclic alkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, and 1,1,2-trimethylpropyl. Examples of cyclic alkyl include cyclopropyl, cyclobutyl, cyclopentyl and cyclohexyl.

In this specification, the term “cycloalkyl” refers to a saturated monocyclic or poly-cyclic alkyl having 3 to 12 carbons.

In this specification, the term “alkenyl” refers to a straight chain, branched or cyclic alkenyl with one or more double bonds. Preferably the alkenyl is a C₂ to C₂₀ alkenyl, more preferably C₂ to C₆ alkenyl. Examples of alkenyl include vinyl, allyl, 1-methylvinyl, butenyl, isobutenyl, 3-methyl-2-butenyl, 1-pentenyl, cyclopentenyl, 1-methylcyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

In this specification, the term “alkynyl” refers to a radical of a straight chain, branched or cyclic alkynyl with one or more triple bonds, preferably a C₂ to C₂₀ alkynyl, more preferably a C₂ to C₆ alkynyl.

In this specification, the term “aryl” used either alone or in compound words such as “arylalkyl”, refers to a radical of a single, polynuclear, conjugated or fused aromatic hydrocarbon or aromatic heterocyclic ring system. Examples of aryl include phenyl, naphthyl and furyl. When the aryl comprises a heterocyclic aromatic ring system, the aromatic heterocyclic ring system may contain 1 to 4 heteroatoms independently selected from N, O and S and up to 9 carbon atoms in the ring.

In this specification the term “arylalkyl” refers to an alkyl substituted with an aryl group. An example of arylalkyl is benzyl.

The present invention relates to complexes of the formula (1):

[Cu(η²-L¹)₂L₂]^{p} (1)

wherein “η²-L¹” is a bidentate ligand of the formula L¹:

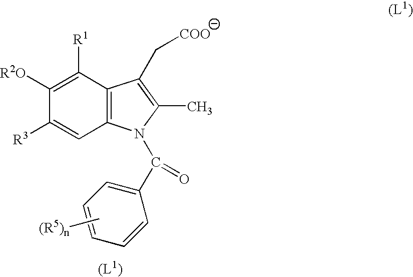

wherein:

R¹ is H or halo (i.e., Cl, F, Br or I);

R² is H; a C₁ to C₆ alkyl, an alkenyl or an alkynyl, where the C₁ to C₆ alkyl, alkenyl or alkynyl may be optionally substituted; or

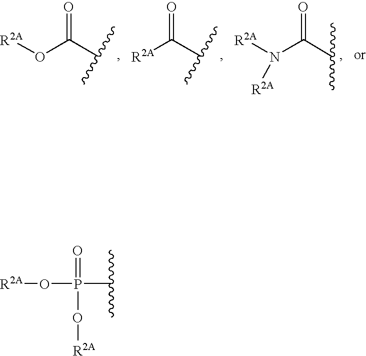

wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;

R³ is H or halo;

each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted;

n is 1, 2, 3, 4 or 5;

each L is independently selected and is a monodentate ligand; and

p is the charge of the complex.

As used in this specification including the claims, by a “bidentate ligand” is meant a ligand having two co-ordination bonds to a metal atom. Bidentate ligands include unsymmetric bidentate ligands with one weaker and one relatively stronger bond to the metal atom. By a “monodentate ligand” it is meant a ligand having a single co-ordination bond with a metal atom.

When R² is a C₁ to C₆ alkyl, an alkenyl or an alkynyl, the C₁ to C₆ alkyl, alkenyl or alkynyl may be substituted with one or more substituents. The one or more substituents may, for example, be independently selected from the group consisting of halo, —OH, —COOH and —NH₂.

When R^{2A} is a C₁ to C₆ alkyl, an alkenyl, an alkynyl, an aryl, a cycloalkyl or an arylalkyl, the C₁ to C₆ alkyl, alkenyl, alkynyl, aryl, cycloalkyl or arylalkyl may be substituted with one or more substituents. The one or more substituents may, for example, be independently selected from the group consisting of halo, —OH, —COOH and —NH₂.

When R⁵ is —CH₃, —OCH₃, —SCH₃ or —CH₂CH₃, the —CH₃, —OCH₃, —SCH₃ or —CH₂CH₃ may be substituted with one or more substituents. The one or more substituents may, for example, be independently selected from the group consisting of halo, —OH, —COOH and —NH₂—.

Typically n is 1, 2 or 3, and each R⁵ is independently selected from I, Br, Cl, or F. In some embodiments, n is 1, 2 or 3 and each R⁵ is independently selected from Cl and Br.

L¹ may be Indo.

L may be a charged or uncharged monodentate ligand. When each L is a neutral ligand, the complex of formula (1) is neutral in charge (i.e., p is 0). However, if L is an anionic ligand, the complex of formula (1) will be charged. In some embodiments, p is 1- or 2-.

The complex of formula (1) may be in solution, or may be in the form of a solid. Crystals of a complex of formula (1) may include solvents of crystallisation, and crystals of a complex of formula (1) incorporating solvents of crystallisation fall within the scope of the present invention. Crystals of a complex of formula (1) may also include waters of crystallisation. Water molecules are present as an impurity in all non-aqueous solvents. Crystals of a complex of formula (1) including waters of crystallisation fall within the scope of the prevent invention.

If L is an anionic ligand, a solid of the complex of formula (1) will include cations that are counterions to the anionic complexes. Such solids, include solids having the following formulae:

Y[Cu(η²-L¹)₂L₂] (1a)

and

Y′₂[Cu(η²-L¹)₂L₉] (1b)

wherein η²-L¹ and L are as defined above for formula (1), Y is a counterion having a 2+ charge and Y′ is a counterion having a 1+ charge.

The present inventors have found that complexes of formula (1) cause less adverse gastrointestinal effects than the administration of an equimolar amount of the group of the formula L¹ in the form of the free compound L¹H. The inventors have also found that complexes of formula (1) cause less adverse gastrointestinal effects than the administration of an equimolar amount of L¹ in the form of a mononuclear copper complex containing one or more monodentate ligands of formula L¹.

Mononuclear copper complexes with one or more monodentate ligands of formula L¹ include complexes of the formula (2):

[Cu(η¹-L¹)ₘL_{q}]^{p} (2)

wherein “η¹-L¹” is a monodentate ligand of the formula L¹:

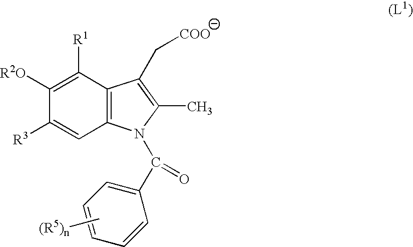

wherein:

R¹ is H or halo (i.e., Cl, F, Br or I);

R² is H; a C₁ to C₆ alkyl, an alkenyl or an alkynyl, where the C₁ to C₆ alkyl, alkenyl or alkynyl may be optionally substituted (for example, with one or more substituents independently selected from the group consisting of halo, —OH, —COOH and —NH₂); or

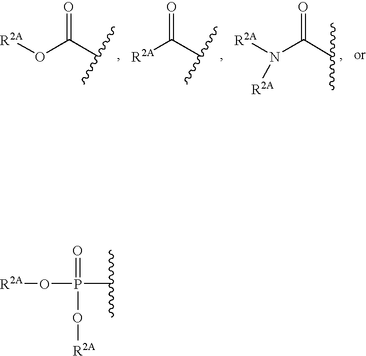

wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted (for example, with one or more substituents independently selected from the group consisting of halo, —OH, —COOH and —NH₂);

R³ is H or halo;

each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted (for example, with one or more substituents independently selected from the group consisting of halo, —OH, —COOH and —NH₂);

n is 1, 2, 3, 4 or 5;

each L is independently selected and is a monodentate ligand or polydentate ligand, m is an integer from 1 to 6, q is 0 or an integer from 1 to 5; and

p is the charge of the complex.

When each L is a monodentate ligand, m+q=4, 5 or 6. Complexes of formula (2) include the complex [Cu(η¹-Indo)₂(Py)₃] where “Py” is pyridine.

The present inventors have found that the oral administration of a complex of formula (1) causes less adverse gastrointestinal effects than the oral administration of an equimolar amount of the ligand L¹ in the form of a complex of the formula (2).

The present inventors have found that the adverse gastrointestinal effects associated with some metal complexes of indomethacin are caused at least in part by the release of some of the indomethacin from the complex. Metal complexes of indomethacin are typically administered to patients in the form of a pharmaceutical composition containing the complex. Indomethacin may be released from the complex during the manufacture of the pharmaceutical composition, during storage of the pharmaceutical composition, or after the complex is administered to the human or animal patient. The present inventors have found that the ligand L¹ is more tightly bound in complexes of formula (1) than in complexes of formula (2), and thus the ligand L¹ is less readily released from a complex of formula (1) compared to complexes of formula (2). The present inventors have found that the oral administration of complexes of formula (2) is associated with similar gastrointestinal side effects to the oral administration of an equimolar dose of L¹ in the form of the free compound L¹H.

The present inventors have further found that complexes of formula (1) are formed when copper(II) indomethacin complexes are formed using strong donor ligands. Complexes of formula (1) may for example be formed using the ligand pyrrolidine. Other ligands having a similar donor strength to, or a greater donor strength than, pyrrolidine also form complexes of formula (1). In some embodiments of the present invention, one or both of the ligands L in the complex of formula (1) is a ligand containing an N-heterocyclic group. In some embodiments, one or both of the ligands L is a ligand containing a pyrrolidine, imidazole, pyrrole, pyrazole, pyridazine, pyrimidine or pyrazine ring. In some embodiments, one or both of the ligands L is pyrrolidine, substituted pyrrolidine (e.g. alkyl-substituted pyrrolidine such as pyrrolidine substituted with 1, 2, 3, 4 or more C₁₋₆ alkyl substituents), proline, substituted proline (e.g. proline substituted with 1, 2, 3 or more C₁₋₆ alkyl substituents), imidazole, substituted imidazole (e.g. imidazole substituted with 1 or 2 C₁₋₆ alkyl substituents), pyrrole, substituted pyrrole (e.g. pyrrole substituted with 1, 2, 3 or 4 C₁₋₆ alkyl substituents), pyrazole, substituted pyrazole (e.g. pyrazole substituted with 1, 2, 3 or 4 C₁₋₆ alkyl substituents), pyridazine, substituted pyridazine (e.g. pyridazine substituted with 1, 2, 3 or 4 C₁₋₆ alkyl substituents), pyrimidine, substituted pyrimidine (e.g. pyrimidine substituted with 1, 2, 3 or 4 C₁₋₆ alkyl substituents), pyrazine, substituted pyrazine (e.g. pyrazine substituted with 1, 2, 3 or 4 C₁₋₆ alkyl substituents), 4-picoline, 3-picoline, 2-picoline, nicotinamide or nicotinic acid. In some embodiments, one or both of the ligands L is imidazole or an imidazole derivative such as substituted imidazole or a ligand containing an imidazole ring (e.g. benzimidazole). In some embodiments, one or both of the ligands L is a pyridine derivative such as 4-picoline, 3-picoline, 2-picoline, nicotinamide or nicotinic acid. In some embodiments, one or both of the ligands L is an amine, e.g. NH₃ or an organic amine (e.g. diethylamine), an alcohol or an amide (e.g. diethylacetamide), or another ligand that is a strong donor such as triethylphosphate.

In some embodiments, L is a solvent having a solvent donor number of about 30 or greater.

For a complex to be administered to a human or animal, L is preferably a pharmaceutically acceptable ligand. By a “pharmaceutically acceptable ligand” it is meant a ligand that does not cause any or a substantial adverse reaction when the complex is administered to a human or animal patient. However, complexes of the formula (1) where one or more L is not a pharmaceutically acceptable ligand fall within the scope of the present invention. Such complexes may be used, for example, as an intermediate in the preparation of complexes of formula (1) where each L is a pharmaceutically acceptable ligand.

Complexes of formula (1) may, for example, be prepared by direct reaction of the appropriate ratios of a compound of the formula L¹H where L¹ is as defined above and a copper salt such as copper(II) acetate in a solvent having a solvent donor number of about 30 or greater, the solvent forming the ligand L in the resulting complex. Complexes of formula (1) may also be prepared by adding a solvent having a solvent donor number of about 30 or greater, or adding a ligand that is not a solvent but has a similar donor strength to a solvent having a solvent donor number of about 30 or greater, to a solution of Cu(II) and L¹ in a weaker donor solvent.

Complexes of formula (1) can also be prepared by re-crystallisation of a dinuclear complex, such as [Cu₂(Indo)₄(DMF)₂], in a solvent having a solvent donor number of about 30 or greater, such as pyrrolidine, or in a solvent containing a ligand that is a strong donor.

Complexes of formula (1) can also be prepared by adding a solution of Cu(II) to a solution containing the two ligands L and L¹, for example, adding the solution of Cu(II) dropwise with stirring as described in Example 3.

One or more embodiments of the complexes of formula (1) are more lipophilic than compounds of the formula L¹H and thus may be more easily absorbed through membranes and taken up by tissues locally. The complexes of formula (1) may, therefore, also be more readily absorbed than compounds of the formula L¹H when administered topically.

The composition of the present invention comprises a complex of formula (1) together with a pharmaceutically acceptable carrier. As used herein, a “pharmaceutically acceptable carrier” is a pharmaceutically acceptable solvent, suspending agent or vehicle for delivering the complex to a human or animal. The carrier may be liquid or solid and is selected with the planned manner of administration in mind. The carrier is “pharmaceutically acceptable” in the sense of being not biologically or otherwise undesirable, i.e., the carrier may be administered to a human or animal along with the complex without the carrier causing any or a substantial adverse reaction.

As used herein, the term “therapeutically effective amount” means an amount effective to yield a desired therapeutic response, for example, to treat an inflammatory condition. The specific “therapeutically effective amount” of the metal complex utilised in a method embodied by the present invention will vary with such factors as the particular condition being treated, the physical condition age and weight of the human or animal, the type of animal being treated, the duration of the treatment, the nature of concurrent therapy (if any), and the specific composition and complex employed. The dosage administered and route of administration will be at the discretion of the attending, clinician or veterinarian and will be determined in accordance with accepted medical or veterinary principles. For instance, a low dosage may initially be administered which is subsequently increased at each administration following evaluation of the response of the subject. Similarly, the frequency of administration may be determined in the same way, that is, by continuously monitoring the response of the subject and modifying the interval between dosages.

The metal complex is typically administered to the human or animal by administering a composition containing the complex. The complex may be administered by any route or mode suitable for the disease or condition being treated. The metal complex may also be administered alone or co-administered in combination with one or more active agents conventionally used in the treatment of inflammation. By “co-administered” is meant simultaneous administration in the same composition or different compositions by the same of different routes, or sequential administration by the same or different routes. By “sequential” administration is meant one is administered one after the other. Such conventional agents includes both metal and non-metal based drugs.

As described in the applicant's co-pending International Patent Application filed 24 Mar. 2006 entitled “Methods for the prophylaxis or treatment of carcinoma” claiming priority from Australian Provisional Application No. 2005901463, one or more embodiments of metal complexes of the present invention and compositions incorporating them may also be used in the prophylaxis or treatment of carcinomas such as one or more carcinomas selected from the group consisting of basal cell carcinomas, squamous cell carcinomas, melanoma, colon cancer, colorectal cancer, breast cancer, lung cancer and other cancers of the epithelium, and the contents of the International Patent Application is hereby incorporated by cross-reference in its entirety.

As will be understood, the use of one or more embodiments of a metal complex of the present invention in combination with other anti-inflammatory or anti-cancer drug may enhance the effectiveness of the other drug. In the prophylaxis or treatment of carcinoma, this may include both carcinomas that are responsive to treatment by the other drug and carcinomas that are otherwise resistant to the other drug.

In particularly preferred embodiments a composition embodied by the invention may be formulated as described in International Application No. PCT/AU2005/000442 filed 30 Mar. 2005, the contents of which is incorporated herein by cross-reference in its entirety. As described in PCT/AU2005/000442, a formulation having a colloidal structure or which forms a colloidal structure post administration is particularly desirable for administration of metal complexes. Examples of suitable compositions having a colloidal structure or which form a colloidal structure upon, or following administration, are exemplified in PCT/AU2005/00042 and any suitable such formulations for the selected mode of administration may be utilised in methods embodied by the present invention. Formation of the colloidal structure can for instance occur when the composition contacts an aqueous biological fluid in the human or animal body, for example, on contact with an aqueous fluid in the digestive tract.

A composition has a colloidal structure if it comprises a colloidal system. A colloidal system is a system in which particles of a colloidal size of any nature (eg., solid as liquid or gas) are dispersed in a colloidal phase of a different composition or state. In particularly preferred embodiments, the composition comprises micelles in an aqueous carrier or is an oil-in-water emulsion, or forms micelles or an oil-in-water emulsion when the composition is administered to a human or animal body.

Without wishing to be limited by theory, it is believed the colloidal structure protects the metal complex from interaction with acids or other compounds which would otherwise interact with the complex to cause the complex to dissociate. It is also believed the colloidal structure reduces the extent to which some compounds present in the composition are able to interact with the complex, e.g. during storage of the composition, that may cause the complex to dissociate. When such a composition is administered to a subject, the colloidal structure may limit the extent to which some compounds that come into contact with the composition after it is administered are able to interact with the complex and which cause the complex to dissociate before it is absorbed. For such compositions administered orally, the colloidal structure may limit the extent to which compounds present in stomach acid are able to interact with the complex to cause the complex to dissociate before it is absorbed through the gastrointestinal tract. Similarly, for compositions administered by other routes, the colloidal structure may limit the extent to which compounds that come into contact with the composition after it is administered, e.g. strong chelators of Cu(II), such as peptides, or reductants of Cu(II), such as thiol-containing biomolecules, are able to interact with the complex to cause the complex to dissociate. As indicated above, some compositions may not have a colloidal structure but will be formulated such that when administered to a human or animal body by the intended route of administration, a colloidal structure is formed. For example, in some embodiments, the composition is immiscible with water, and is thus immiscible with aqueous biological fluids whereby a colloidal system is thereby formed.

Preferably, the colloidal structure is maintained for a sufficient time after administration of the composition for the majority, for example more than 70%, 80% or 90%, of the metal complex, to be absorbed by the body as a metal complex.

Oils for use in the compositions include pharmaceutically acceptable vegetable or mineral oils. Suitable oils include, but are not limited to: triglycerides, particularly medium chain triglycerides, combinations of medium chain and long-chain triglycerides, combinations of triglycerides with fish oil; vegetable oils, such as, soya oil, safflower oil and sunflower oils; isopropyl myristate; and paraffins. Such oils are suitable for use in compositions for oral, injectable, or topical administration.

When the composition comprises micelles in an aqueous carrier, the composition will typically further comprise one or more surfactants for formation of the micelles. Any surfactants may be used that are capable of forming micelles in the aqueous carrier, are pharmaceutically acceptable when administered by the intended route of administration, and which substantially do not interact with the metal carboxylate complex to cause dissociation from the metal when the composition is stored in the absence of light. Suitable surfactants for use in compositions for oral or topical administration of metal complexes of the invention include, but are not limited to, the sorbitan fatty acid ester group of surfactants. Such surfactants comprise mono-, tri-, or partial esters of fatty acids such as oleic, lauric, palmic and stearic acids, and include sorbitan trioleate (Span 85), sorbitan monooleate (Span 80), sorbitan tristearate (Span 65), sorbitan monostearate, (Span 60), sorbitan monopalmitate (Span 40), and sorbitan monolaurate (Span 20).

Other suitable surfactants include the macrogol (polyoxyethylene) esters and ethers. These surfactants include, but are not limited to, the caster oil polyoxyethylene group of surfactants, such as Termul 1284 and caster oil ethoxylate. Further surfactants in this class include the polyoxyethylene sorbitan fatty acid esters group of surfactants, including polyoxyethylene (20) sorbitan monolaurate (Tween 20), polyoxyethylene (4) sorbitan monolaurate (Tween 21), and polyoxyethylene (20) sorbitan monooleate (Tween 80).

Other suitable surfactants include the block copolymers based on ethylene oxide and propylene oxide such Poloxamer 124 (Pluronic L44 NF), Poloxamer 188 (Pluronic F68 NF), Poloxamer 331 (Pluronic L101 NF), and Poloxamer 407 (Pluronic F127 NF). Suitable surfactants also include the polyethylene glycol fatty acid esters (PEG esters) group of surfactants. Such surfactants comprise mono-, tri-, or partial esters of fatty acids such as oleic, lauric, palmic, oleic, and stearic acids, including but not limited to PEG 200 monolaurate, PEG 300 dilaurate, ethylene glycol distearate, PEG 300 monooleate, PEG 400 monooleate, PEG 350 monostearate, PEG 300 monostearate, PEG 400 Monostearate, PEG 600 Monostearate, PEG 1000 monostearate, PEG 1800 monostearate, PEG 6500 monostearate, PEG 400 mono-iso stearate, PEG 600 mono-iso-stearate, PEG 200 dilaurate, PEG 600 distearate, PEG 6000 distearate, PEG 200 distearate, PEG 300 distearate, and PEG 400 distearate.

A composition as described herein may also optionally further comprise one or more solvents, co-solvents or solubilising components for increasing the solubility of the metal carboxylate complex in the composition. The solvent or co-solvent may, for example, be tetraglycol (IUPAC name: 2-[2-[(tetrahydro-2-furanyl)methoxy]ethoxy]ethanol; other names: 2-[2-(tetrahydrofurfuryloxy)ethoxy]ethanol; tetrahydrofurfuryldiethyleneglycol ether) or other glycofurols (also known as tetrahydrofurfurylpolyethyleneglycol ethers), polyethylene glycols, glycerol, propylene glycol, butyl glycol or other pharmaceutically acceptable glycol. Further suitable co-solvents include ethoxylated alcohols and aromatic alcohols including cetyl alcohol, stearyl alcohol, lauryl alcohol, benzyl alcohol, and ethoxydiglycol. An example of a solubilising component is a polyvinylacohol/povidone mixture. The composition may also further comprise a thickener such as Aerosil 200, clay or another inorganic filler.

Suitable viscosity imparting or suspending agents include sorbitol, povidone, soya bean lecithin, cholesterol and egg yolk phospholipid.

Strong chelating ligands such as peptides, certain carboxylate donors, reductants such as vitamins C and E, thiolate groups such as glutathione- or cysteine-containing species, can cause metal carboxylate complexes to dissociate. Accordingly, the compositions preferably do not comprise, or are substantially free of, peptides, carboxylate donors, reductants and thiolate groups. Preferably, the composition is also not strongly acidic or basic as strong acids and bases can cause metal carboxylate complexes to dissociate.

In some embodiments, all of the groups of the formula L¹ present in the composition embodied by the invention are present as part of a complex of formula (1). In other embodiments, some groups of the formula L¹ present in the composition are present in some other form, e.g. in the form of the free compound L¹H, in the form of the ion L¹, as part of a dimer complex containing the ligand L¹ or as part of a complex of formula (2). In such embodiments, typically more than 50%, more typically more than 80%, and even more typically more than 95%, of groups of the formula L¹ present in the composition are present as part of a complex of formula (1).

Preferably, in one or more embodiments of compositions of the invention, more than 80%, preferably more than 90%, and more preferably more than 95%, of the total amount of the copper atom is present in the composition as part of the metal complex, and less than 10% of metal complex dissociates when the composition is stored for 12 months in the absence of light at room temperature (18° C. to 25° C.). The degree of dissociation of the metal complex in the composition can be readily determined by a person skilled in the art using known methods such as EPR spectroscopy.

More generally, the metal complex may be dissolved in the composition or may be present in the composition as a solid. The solid complex may be in the form of a crystal containing solvents of crystallisation and/or waters of crystallisation. When the complex is charged, the complex will be associated with a counter ion.

Compositions useful for administering metal complexes embodied by the invention include those suitable for oral, rectal, nasal, topical (including buccal and sublingual), transdermal, opthalmological, vaginal or parenteral (including subcutaneous, intramuscular, intravenous and intradermal) administration, and for instance, administration by inhalation.

The composition may also conveniently be presented in unit dosage form and may be prepared by methods well known in the art of pharmacy. Such methods include the step of bringing into association the complex with the carrier. Typically the carrier consists of two or more components. In general, the composition of the present invention is prepared by uniformly and intimately bringing into association the complex with the carrier, and then if necessary shaping the product. The complex and the one or more ingredients making up the carrier may be mixed in any order. However, it will be appreciated that the components are mixed in a manner that minimises dissociation of the metal complex during the preparation of the composition.

A composition for oral administration of a metal complex in accordance with an embodiment of the invention may be in the form of a viscous paste, a digestible tablet, a capsule, a chewable composition, or any other form suitable for oral administration. If desired, the composition may be encapsulated in a soft or hard capsule by techniques known in the art. Moreover, the metal complex may be provided in the form of buccal tablets, troches, elixirs, suspensions or syrups. Slow release formulations and formulations for facilitating passage through the environment of the stomach to the small intestines are also well known to the skilled addressee and are expressly encompassed by the invention.

Compositions for oral administration include, for example, a composition containing 2% (w/v) of a complex of formula (1) in CMC solution. Another example of a composition for oral administration is a paste formulation comprising 2% (w/v) of a complex formula (1), one or more glycofurols (e.g. tetraglycol), one or more surfactants, one or more thickeners and a medium chain triglyceride.

A composition for oral use may for instance, also comprise one or more agents selected from the group of sweetening agents such as sucrose, lactose or saccharin, disintegrating agents such as corn starch, potato starch or alginic acid, lubricants such as magnesium stearate, flavouring agents, colouring agents and preserving agents e.g. such as sorbic acid, in order to produce pharmaceutically elegant and palatable preparations.

A chewable composition may for example comprise the complex of formula (1), one or more flavours, a base fomulation, one or more preservatives, one or more pH modifiers, one or more desiccants and one or more fillers. For example, a chewable composition for horses may comprise the complex of formula (1), flavour, the base (comprising pre-gel starch, gelatine, flour and water), and other components including phosphoric acid, salt, sugar, sorbitol and/or glycerol, sorbic acid and/or potassium sorbate, benzoic acid, propionic acid and maltodextrin. A chewable composition for dogs may comprise the complex of formula (1), meat emulsion, an acidulate (e.g. phosphoric acid), one or more antifungal agents (e.g. benzoic acid and sorbic acid), sugar or sugar alcohol, and salt.

A composition of the present invention for topical application may comprise the complex of formula (1) in a conventional oil-in-water emulsion, water-in-oil emulsion, or water-immiscible pharmaceutical carrier suitable for topical application.

Such carriers include for example, lacrilube, cetomacrogol cream BP, wool fat ointment BP or emulsifying ointment BP. Such carriers are in the form of an emulsion or are immiscible with water.

An example of a composition for topical application is a composition comprising 0.5-2% w/w of the complex of formula (1) in an emulsifying cream comprising chlorocresol (4-chloro-3-methylphenol) as a preservative as follows:

| | | |
|---|---|---|
| cetomacrogol emulsifying wax | 15 | g |
| liquid paraffin | 10 | g |
| white soft paraffin | 10 | g |
| chlorocresol | 0.1 | g |
| propylene glycol | 5 | ml |
| purified and cooled water | to 100 | g. |

Another example of a topical composition is a composition consisting of 0.5-2% w/w of the complex of formula (1) in wool fat. This composition is immiscible with water.

Another example of a topical formulation for a metal complex embodied by the invention is as follows.

| | Amount (% by weight |
|---|---|
| Ingredient | of the composition) |
| Oil Phase | |
| Myrj 45 | 10.00 |
| Isopropyl Lanolate | 3.00 |
| Lantrol | 1.00 |
| Modulan | 0.50 |
| Isopropyl myristate | 5.00 |
| Mineral oil | 4.75 |
| Emulan | 3.00 |
| Cetyl alcohol | 0.10 |
| Complex | 0.25 |
| Water Phase | |
| Veegum | 1.00 |
| Water | 66.80 |
| Propylene glycol | 3.00 |
| Methyl paraben | 0.20 |
| | 100.00 |

This composition is an emollient oil-in-water cream. The composition may be prepared by separately preparing the oil phase and water phase by mixing the components of each phase, and then adding the water phase to the oil phase at 65° C. after blending the Veegum into the water. The complex is dissolved in the oil phase prior to emulsification. For a less viscous lotion, the % w/w of Veegum may be reduced to 0.50% to 0.75%.

A yet further example of a composition for topical application to skin is a composition comprising 0.5-2% w/w of the complex in an emulsifying cream with chlorocresol (4-chloro-3-methylphenol) as a preservative as follows:

| Ingredient | Amount | |
|---|---|---|
| cetomacrogol emulsifying wax | 15 | g |
| liquid paraffin | 10 | g |
| white soft paraffin | 10 | g |
| chlorocresol | 0.1 | g |
| propylene glycol | 5 | mL |
| purified and cooled water | to 100 | g |

An example of a composition for rectal administration of a metal complex as described herein such as to infants or for paediatric use may be prepared as follows. Amounts shown are % w/w of the composition.

| Ingredient | Amount |
|---|---|
| One or more metal complexes | 10% |
| having anti-inflammatory activity | |
| Macrogol 400 | 20% |
| Macrogol 4000 | 70% |

This composition is an un-reactive non-greasy, water miscible suppository base which does not ionise in the presence of water. The proportions of the macrogols (ethylene glycol polymers) are determined to provide a melting point of the suppositories which is not higher than 37° C. Allowances are made for volume occupied by the metal complex in each suppository, i.e based on densities of the complex relative to the base.

Typically, the metal complex will constitute about 0.025% to about 20% by weight of a composition embodied by the present invention, preferably about 0.025% to about 20% by weight of the composition, more preferably about 0.1% to about 20% by weight of the composition and most preferably, the complex constitutes about 0.1% to about 10% by weight of the composition.

In some embodiments, a composition of the invention does not comprise any therapeutically active ingredients in addition to the complex of formula (1). In other embodiments, a composition embodied by the invention may include one or more therapeutically active agent(s) in addition to the complex of formula (1). The active agent(s) may for instance be selected from drugs conventionally used for the prophylaxis or treatment of inflammation or other conditions.

Suitable pharmaceutically acceptable carriers and formulations useful in the present invention may for instance be found in handbooks and texts well known to the skilled addressee, such as “Remington: The Science and Practice of Pharmacy (Mack Publishing Co., 1995)” and subsequent update versions thereof, the contents of which is incorporated herein by reference in its entirety.

The human or animal may be any human or animal having a disease or condition in need of treatment by a method embodied by the present invention. The animal is typically a mammal, and may be a non-human primate or non-primate. The mammal may for example be a companion animal such as a dog or cat, or a domestic animal such as a horse, pony, donkey, mule, camel, llama, alpaca, pig, cow or sheep, or a zoo animal. Suitable mammals include members of the Orders Primates, Rodentia, Lagomorpha, Cetacea, Carnivora, Perissodactyla and Artiodactyla.

Typically, the subject will be a dog, primate, or a human being.

The inflammatory condition may for example be rheumatoid arthritis, osteoarthritis, acute musculoskeletal disorders (such as tendonitis, sprains and strains), or lower back pain (commonly referred to as lumbago). The inflammatory condition may also be inflammation, pain or edema following surgical or non-surgical procedures, or any other inflammatory disease or condition responsive to treatment as described herein.

The invention is described further below by reference to a number of non-limiting examples.

### Example 1

### Preparation of bis(η¹-O-Indo)tris(pyridine)copper(II), [Cu(Indo)₂(Py)₃](“Complex 1”) and bis(η²-O,O′-Indo)bis(pyrrolidine)copper(II)-2-pyrrolidine monohydrate, [Cu(Indo)₂(Pyrro)₂].2Pyrro.H₂O (“Complex 2”).

### Experimental

### Chemicals

IndoH of pharmaceutical grade (Sigma-Aldrich) was used as received. [Cu₂(Indo)₄(DMF)₂] was provided by Biochemical Veterinary Research Pty Ltd. (BVR) and was purified by two recrystallisations from DMF. [Cu₂(OAc)₄(OH₂)₂] was obtained from Univar (99% purity). All of the other chemicals were of analytical grade (Sigma-Aldrich).

For comparative purposes, the complexes [Cu₂(Indo)₄(DMA)₂], [Cu₄(Indo)₂(THF)₂], [Cu₂(Indo)₄(ACN)₂] and [Cu₂(Indo)₄(Py)₂] were prepared as reported previously (Preparation and Characterization of Dinuclear Copper-Indomethacin Anti-Inflammatory Drugs. Morgan, Y. R.; Turner, P.; Kennedy, B. J.; Hambley, T. W.; Lay, P. A.; Biffin, J. R.; Regtop, H. L; Warwick, B. Inorg. Chim. Acta 2001, 324, 150-161).

The structures of the Cu-Indo dimers [Cu₂(Indo)₄(L)₂] (where L=DMA, THF, ACN or Py), Complex 1, Complex 2, and their solvent ligands is set out below:

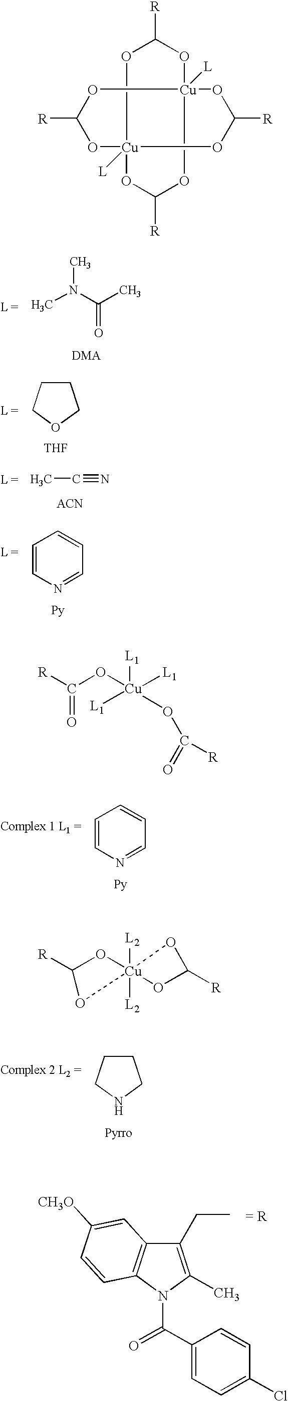

### Bis(η¹-O-Indo)tris(pyridine)copper(II), [Cu(Indo)₂(Py)₃] (Complex 1)

Crystals of Complex 1 were prepared as reported in Preparation and Characterization of Dinuclear Copper-Indomethacin Anti-Inflammatory Drugs. Morgan, Y. R.; Turner, P.; Kennedy, B. J.; Hambley, T. W.; Lay, P. A.; Biffin, J. R.; Regtop, H. L; Warwick, B. Inorg. Chim. Acta 2001, 324, 150-161.

Blue tabular crystals were grown by recrystallisation of [Cu₂(Indo)₄(DMF)₂] twice from mixtures of pyridine and ethanol with 1:1 and 2:5 volume ratios, respectively. Anal. Found: C, 62.38; H, 4.67; N, 7.47; Cu, 6.66%. Calc. for CuC₅₃H₄₅Cl₂N₅O₈: C, 62.75; H, 4.47; N, 6.91; Cu, 6.26%.

### Bis(η²-O,O′-Indo)bis(pyrrolidine)copper(II)-2-pyrrolidine monohydrate, [Cu(Indo)₂(Pyrro)₂].2Pyrro.H₂O (Complex 2)

X-ray diffraction quality crystals that consisted of pale blue plates were grown by recrystallisation of [Cu₂(Indo)₄(DMF)₂] in pyrrolidine as the solvent. Anal. Found: C, 59.91; H, 6.32; N, 7.84; Cu, 6.01%. Calc. for CuC₅₄H₆₈Cl₂N₆O₉: C, 60.15; H, 6.36; N, 7.80; Cu, 5.84%.

### Physical Measurements

### Elemental Microanalyses.

Copper analyses were performed with a Varian AA-800 air-acetylene flame atomic absorption spectrophotometer. The C, H, N microanalyses were performed by the Department of Chemistry, University of Otago.

### Infrared Spectroscopy

Fourier transform IR spectra were acquired from samples within pressed disks of KBr matrix on a Bio-Rad Win-IR FTS-40 infrared spectrometer (400-4000 cm⁻¹).

### UV-Vis Spectroscopy.

Diffuse-reflectance solid-state UV-Vis spectra were recorded using a Varian Cary 1E spectrophotometer. UV-Vis spectra of solutions were obtained in 1-cm quartz cells in a Hewlett-Packard 8452A diode-array (190-820 nm) or a Varian Cary 5E UV-VIS-NIR spectrophotometer. Each complex was dissolved in the same solvent as its solvent ligand.

### X-Band Electron Paramagnetic Resonance Spectroscopy

X-band (˜9.5 GHz) EPR spectra of powdered and solution samples of the complexes were acquired using a Bruker EMX EPR spectrometer equipped with a standard ER4120X-band cavity, EMX 035M NMR gaussmeter, EMX 032 field controller, EMX 081 magnet power supply, Bruker EMMX 048T microwave bridge control, and BVT2000 variable temperature unit.

### Magnetic Susceptibility

Room-temperature magnetic susceptibilities (Xg) and magnetic moments (μ_{eff}) were measured with a Sherwood Scientific magnetic susceptibility balance. Iron(II) ammonium sulfate hexahydrate was used as a standard for calibration of the instrument.⁶ The value of X_{D} was obtained by summing the atomic diamagnetism of all diamagnetic atoms present in [Cu₂(Indo)₄(L)₂], or [Cu(Indo)₂(L)₃] and [Cu(Indo)₂(L)₂] and a small constitutive correction (ε) for specific electronic characteristics, e.g. π-bonds.⁶

### X-Ray Powder Diffraction

X-ray powder diffraction patterns were collected at room temperature using Cu Kα radiation with a Shimadzu Lab XRD-6000 diffractometer with divergence and anti-scatter slits of 0.5 mm, and receiver and detector slits of 0.15 and 0.6 mm, respectively. These data were collected over the range 5.0-40.0° in steps of 0.02° in 2θ, and a count time per step of 15.0 s. Profiles were fitted using the La-Bail method implemented in the program, Rietica. In these analyses, cell parameters were initially set equal to those reported for [Cu₂(Indo)₄(DMF)₂] and refined using a non-linear least-squares method.

### X-Ray Crystallographic Analyses

All structures were obtained from diffraction data collected at low temperatures (150-170 K) on a Bruker SMART 1000 diffractometer equipped with an Oxford Cryosystems Cryostream, using graphite-monochromated Mo Kα radiation generated from a sealed tube. Crystals of Complex 1 and Complex 2 were each attached with Exxon Paratone N, to a short length of fibre supported on a thin piece of Cu wire inserted in a Cu mounting pin.

These crystals were quenched in a cold gas (N₂) stream when mounted on the diffractometer. The SMART 1000 data integration and reduction were performed with SAINT and XPREP,⁷ and subsequent computations were performed with TEXSAN.⁸ For Complex 1 WINGX,⁹ and the XTAL¹⁰ graphical user interface were also used. A Gaussian absorption correction was applied to the data for Complex 1 and Complex 2.^{7,11} The structure for Complex 2 was solved in the space group P1(#2) by direct methods with SIR97,¹² and extended and refined with SHELXL-97.¹³ In all cases, data reduction included the application of Lorentz and polarization corrections.

Cell constants for Complex 1 were obtained from a least-squares refinement against 995 reflections located between 5.35 and 52.34° 2θ. Data were collected at 150(2) K and 295(2) K with co-scans to 56.48° 2θ. The intensities of 291 standard reflections that were recollected at the end of the experiment did not change significantly during the data collection. The structure was solved in the space group P2₁/c(#14) by direct methods with SIR97,¹⁴ and extended and refined with SHELXL-97.¹⁵ The asymmetric unit contains a five-coordinate Cu(II) complex comprised of two indomethacin ligands and three pyridine ligands, together with two pyridine solvent molecules and a water molecule. The water molecule is involved in hydrogen bond interactions between the carboxylate O(2) of one indomethacin ligand, and the O(6) carboxylate oxygen of the second indomethacin ligand. The N(7) pyridine molecule is centred on an inversion site, and is accordingly disordered with N(7) and C(61) sharing the same sites with equal occupancies. In general the non-hydrogen atoms were modelled with anisotropic displacement parameters; isotropic displacement parameters were used for the disordered pyridine solvate molecule. The water hydrogens were located in a final difference map, and a riding atom model was used for all of the hydrogen atoms.

Cell constants for Complex 2 were obtained from a least-squares refinement against 838 reflections located between 5.66 and 52.04° 2θ. Data were collected at 150(2) K with ω-scans to 56.74° 2θ. The intensities of 60 standard reflections recollected at the end of the experiment did not change significantly during the data collection. The asymmetric unit contains half of a complex molecule with the metal ion located on an inversion site. The non-hydrogen atoms were modelled with anisotropic displacement parameters and in general a riding atom model was used for hydrogen atoms. The pyrrolidine hydrogen site H(2N) was located and the atom was modelled with an isotropic displacement parameter. The complex may be described as a strongly tetragonally distorted octahedral with two equivalent unsymmetric Indo chelate rings (Cu—O(1) is 1.9719(14) Å and Cu—O(2) is 2.5696(16) Å).

Crystallographic data and structure refinement parameters for Complex 1 and Complex 2 in are summarised Table 1.

### Results

### Synthesis of Dinuclear and Mononuclear Copper Complexes

The precipitation of dinuclear [Cu₂(μ-Indo)₄(Py)₂] or mononuclear [Cu(η¹-Indo)₂(Py)₃] is sensitive to both the ratio of ethanol and pyridine and the time taken for recrystallisation (the monomer that initially precipitates slowly dissolves and converts to the less soluble dimer), with the monomeric complex being the dominant species in solution. Elemental analyses of all the Cu(II)-Indo complexes revealed that the resulting complexes contained varying amounts of solvent molecules, which act as ligands bound to the Cu(II), and/or solvents of crystallisation, as is demonstrated by the crystal structural studies and this is expected to be the case for most monomers of both types described herein. The mononuclear Pyrro complex, however, forms exclusively in both the solid-state and solution, with no evidence of a dimer in either state.

### UV-Vis Spectroscopy

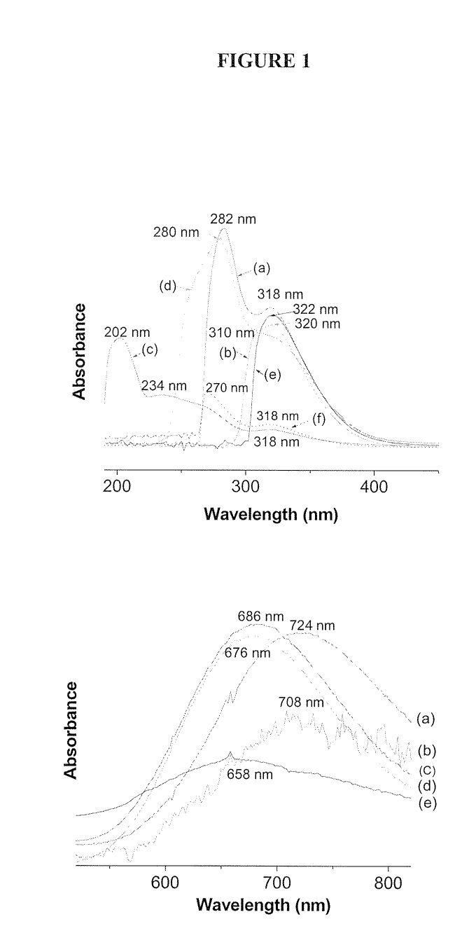

A summary of UV-Vis absorption spectral data for [Cu₂(Indo)₄L₂] (L=DMA, THF or ACN), [Cu(Indo)₂(Py)₃] (Complex 1) and [Cu(Indo)₂(Pyrro)₂] (Complex 2) are given in Table 2. Electronic absorption spectra from solutions of monomers and dimers are given in FIG. 1. Properties of the solvents are listed in Table 3. In FIG. 1, the loss of intensity of the absorbance in the UV region in some solvents was due to the absorbance of the solvent (see Table 3).

The solid-state UV-Vis spectra of both monomer and dimer complexes exhibited a low-energy band centered at about 671 to 728 nm (band I) and more intense higher-energy band at around 345 nm (band II). There are no clear differences between the dinuclear and mononuclear Cu-Indo complexes in the solid-state UV-Vis spectra.

TABLE 1
Crystal data and structure refinement parameters for Complex 1 and
Complex 2

| | Complexes | |
|---|---|---|
| | Complex 1 | Complex 2 |
| Formula of the Refinement | C_{60·50}H_{50.50}Cl₂CuN_{6.50}O₉ | C₄₆H₄₈Cl₂CuN₄O₈ |
| Model | | |
| Model Molecular Weight | 1151.05 | 919.32 |
| Crystal color and habit | blue, blade | pale blue, tabular |
| Crystal system | Monoclinic | Plate |
| Crystal size (mm) | 0.571 × 0.132 × 0.036 | 0.269 × 0.134 × 0.032 |
| Space group | P2₁/c (#14) | P1 (#2) |
| Unit cell dimensions | | |
| a (Å) | 13.0694(12) | 13.420(4) |
| b (Å) | 44.434(4) | 14.845(5) |
| c (Å) | 9.8730(9) | 5.3760(17) |
| α (°) | | 96.948(5) |
| β (°) | 94.137(2) | 91.524(5) |
| γ (°) | | 101.892(5) |
| V (Å³) | 5718.6(9) | 1038.9(6) |
| D_{calc} (g cm⁻³) | 1.337 | 1.469 |
| Z | 4 | 1 |
| λ(Mo Kα) (Å) | 0.71073 | 0.71069 |
| μ (Mo Kα) (mm⁻¹) | 0.538 | 0.716 |
| T(GAUSSIAN)_{min,max} | 0.875, 0.982 | 0.858, 0.977 |
| 2θₘₐₓ (°) | 56.48 | 56.74 |
| Index ranges | −16 ≦ h ≦ 16, −58 ≦ k ≦ 58, | −17 ≦ h ≦ 17, −19 ≦ k ≦ 19, |
| | −12 ≦ l ≦ 12 | −7 ≦ l ≦ 7 |
| N | 48 866 | 9421 |
| N_{ind} | 12977 (R_{merge} 0.0542) | 4745 (R_{merge} 0.0449) |
| N_{obs} | 9014 [I > 2 σ (I)] | 3278 [I > 2 σ (I)] |
| Nᵥₐᵣ | 701 | 283 |
| Residuals R₁(F), wR₂(F²) | 0.0499, 0.1156^{a,b} | 0.0388, 0.0870^{a,c} |
| Goodness-of-fit on F² | 1.294 | 0.900 |
| Residual extrema (e Å⁻³) | −0.445, 0.711 | −0.274, 0.383 |

^{a}R₁ = Σ||Fₒ| |F_{c}||/Σ|Fₒ| for Fₒ > 2 σ (Fₒ); wR2 = (Σw(Fₒ²⁻ F_{c}²)²/Σ(wF_{c}²)²)^{1/2} all reflections.
^{b}w = 1/[σ²(Fₒ²) + (0.0400P)² + 0.5000P], where P = (Fₒ² + 2F_{c}²)/3.
^{c}w = 1/[σ²(Fₒ²) + (0.0396P)² + 0.0000P] where P = (Fₒ² + 2F_{c}²)/3.

TABLE 2
UV-Vis data of solutions and solid samples (diffuse-reflectance
spectra) of Cu-Indo complexes and IndoH.

| | Solution | Solid |
|---|---|---|
| Compounds | λₘₐₓ (nm), εₘₐₓ (M⁻¹ cm⁻¹) | λₘₐₓ (nm) |
| [Cu₂(Indo)₄(DMA)₂] | 282 s, (354 × 10²); 318 | 341 s, 728 s |
| | sh (225 × 10²); 724 s, (405)^{a} | |
| [Cu₂(Indo)₄(THF)₂] | 280 s, (369 × 10²); 320 | 348 s, 728 s |
| | sh (215 × 10²); 676 s, | |
| | (437)^{b} | |
| [Cu₂(Indo)₄(ACN)₂] | 202 s, (181 × 10³); 234 | 345 s, 684 s |
| | sh (863 × 10²); 318 sh | |
| | (285 × 10²); 686 s, (570)^{c} | |
| [Cu(Indo)₂(Py)₃] | 322 s, (134 × 10²); 658 s, (125)^{d} | 342 s, 671 s |
| [Cu(Indo)₂(Pyrro)₂] | 310 s (105 × 10²); 708 s, (153)^{e} | |
| IndoH | 270 s, (155 × 10²); 318 sh | |
| | (637 × 10)^{f} | |

^{a}In DMA.
^{b}In THF.
^{c}In ACN.
^{d}In Py.
^{e}In Pyrro.
^{f}In DMF.

TABLE 3
Some properties of solvents^{16,17}

Solvent

| | ACN | THF | DMA | Py |
|---|---|---|---|---|
| UV Cutoff (nm) | 190 | 215 | 268 | 305 |
| Donor number D_{N} | 14.1 | 20.0 | 26.6 | 33.1 |

In order to avoid ligand-exchange reactions with the solvent that could lead to structural changes, each Cu-Indo complex was dissolved in the same solvent as that coordinated to the Cu(II) centres for solution spectra. The solution-state UV-Vis spectra of the complexes show a broad absorption band in the visible region around 650-750 nm (Band I). The value of ε for this band in solution is much higher for dinuclear complexes, than for the mononuclear complexes. Similar behaviour is observed with other mononuclear and dinuclear Cu(II) complexes,^{1,2,18-23} which suggests that is diagnostic for determining whether the complexes are mononuclear or dinuclear in solution. In addition, there were distinctly different positions of the band in the visible region for the two different forms of monomers (Table 2 and FIG. 1), which appears to be diagnostic of the different structures of the monomers in solution.

### Vibrational Spectra

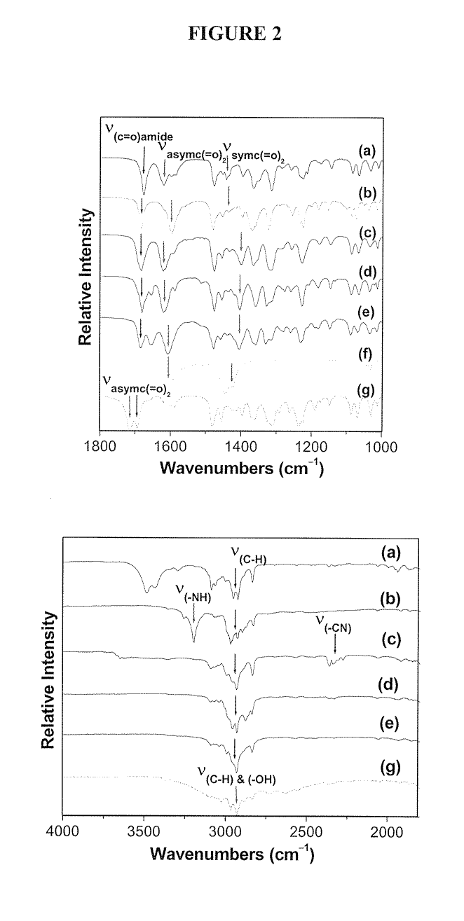

The IR spectral data for the compounds [Cu₂(Indo)₄L₂] (L=DMA, THF or ACN), [Cu(Indo)₂(Py)₃] (Complex 1), [Cu(Indo)₂(Pyrro)₂] (Complex 2), [Cu₂(OAc)₄(OH₂)₂] and IndoH are set out in Table 4 and FIG. 2. All of the complexes exhibit characteristic bands for their ligands in the IR spectra. Features of most interest are bands due to the v_{asym}C( [symbol] O)₂, v_{sym}C( [symbol] O)₂, v_{amide}(C═O) and v_{carbo}(—OH) modes. Depending upon the coordination mode of the carboxylate group, i.e., bridging, monodentate or bidentate, the frequency of the v_{sym}C( [symbol] O)₂, stretching vibrations shift to slightly different positions. The IR spectra for all the complexes exhibit an intense absorption band around 1602-1623 cm⁻¹ (Table 4), due to the v_{asym}C( [symbol] O)₂ vibrational mode, which is at 1716 cm⁻¹ in IndoH. All amide stretching modes v_{amide}(C═O) of these complexes and of IndoH produce strong bands near 1685 cm⁻¹. The band due to the v_{sym}C( [symbol] O)₂ stretch is at a lower frequency ˜1,400 cm⁻¹ in the dimeric species than in the monomeric complexes 1445 and 1437 cm⁻¹, respectively, for complexes 1 and 2. This band is at 1310 cm⁻¹ for IndoH. The Δv values of ca. 200-220 cm⁻¹ for carboxylate bridging ligands in dinuclear [Cu₂(Indo)₄(L)₂] complexes, where Δv=v_{asym}C( [symbol] O)₂−v_{sym}C( [symbol] O)₂, are greater than for those for unidentate coordination of the carboxylates in [Cu(Indo)₂(Py)₃] (178 cm⁻¹) and the unsymmetric bidentate coordination in [Cu(Indo)₂(Pyrro)₂] (160 cm⁻¹).

These band positions of v_{asym}C( [symbol] O)₂, v_{sym}C( [symbol] O)₂ and the Δv values are consistent with those observed for [CU₂(Indo)₄(DMF)₂],^{2,16} Zn-Indo analogs⁵ and other bridging,^{24,29,30} and unidentate^{19-21,26} carboxylate complexes of Cu(II). IndoH displays a very broad, intense v_{carbo} (—OH) stretching absorption in the region of 2500-3300 cm⁻¹. The absence of the v_{carbo} (—OH) absorption bands of the carboxylic acid in the IR spectra of [Cu₂(Indo)₄(L)₂], [Cu(Indo)₂(Py)₃] and [Cu(Indo)₂(Pyrro)₂] is indicative of carboxylate group binding (FIG. 2).

TABLE 4
Room-temperature magnetic moment and IR spectral data for the
complexes and IndoH

| | μ_{eff}^{a} | ν_{amide(C═O)} | ν_{asym(COO)} | ν_{sym(COO)} | |
|---|---|---|---|---|---|
| Compounds | T = 300.6 K | (cm⁻¹) | (cm⁻¹) | (cm⁻¹) | Δν (cm⁻¹) |
| [Cu₂(Indo)₄(DMA)₂ | 1.48 | 1685 | 1602 | 1404 | 198 |
| [Cu₂(Indo)₄(THF)₂] | 1.45 | 1683 | 1622 | 1405 | 217 |
| [Cu₂(Indo)₄(ACN)₂] | 1.37 | 1685 | 1623 | 1402 | 221 |
| [Cu(Indo)₂(Py)₃] | 1.73 | 1679 | 1623 | 1445 | 178 |
| [Cu(Indo)₂(Pyrro)₂] | — | 1684 | 1597 | 1437 | 160 |
| [Cu₂(OAc)₄(OH₂)₂] | — | — | 1618 | 1417 | 201 |
| IndoH | — | 1694 | 1717 | 1310 | 407 |

^{a}This is the value per Cu(II) for the dinuclear complexes.

The IR spectral data for [Cu₂(Ac)₄(OH₂)₂] is included in Table 4 for comparison.

### Magnetic Susceptibility

Room-temperature solid-state effective magnetic moments of the complexes [Cu₂(Indo)₄L₂] (L=DMA, THF or ACN) and [Cu(Indo)₂(Py)₃] (Complex 1) are listed in Table 4. Consistent with anti-ferromagnetic exchange,³¹ the room temperature magnetic moments per Cu for the dimers (μ_{eff}=1.37-1.48 B.M) are similar to those observed for [Cu₂(Indo)₄(DMF)₂] and other dinuclear [Cu₂(RCOO)₄(L)₂] complexes^{2,25,27,32-36} and are somewhat smaller than that expected for the monomeric Complex 1. These observations are due to the singlet ground state and a thermally populated triplet state,^{2,28} the spin interactions occur between the two d⁹ Cu(II) ions via the conjugated π-system of the carboxylate bridges.²⁹ The monomeric Complex 1 has a magnetic moment (μ_{eff}=1.73 B.M) that is typical of a d⁹ spin only (no coupling) mononuclear Cu(II), which is consistent with the EPR results.

### EPR Spectroscopy

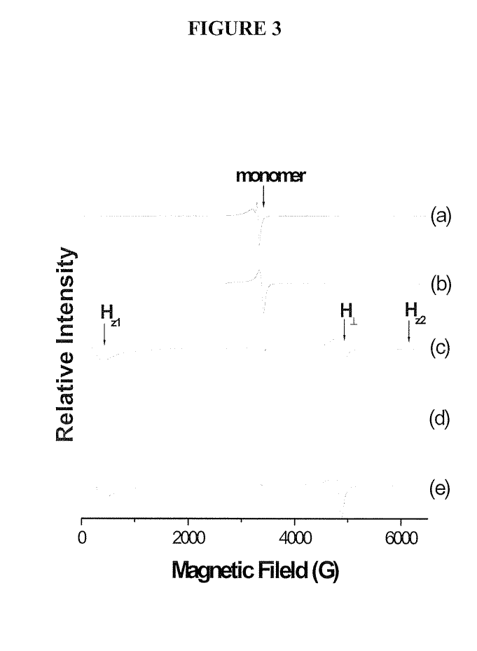

The X-band EPR data for various Cu(II)-Indo complexes in the solid state and solution are summarized in Table 5. The [Cu₂(Indo)₄L₂] (L=DMA, ACN, THF) complexes exhibited distinctive resonances of the S=1 excited state of the dimeric complexes^{1,2} in the X-band EPR spectra (FIG. 3). A small resonance at ˜3300 G in these spectra is due to a trace of a Cu(II) monomer impurity of uncertain structure² in the [Cu₂(Indo)₄L₂] complexes. The EPR spectrum of the monomeric complex, [Cu(Indo)₂(Py)₃], at room temperature (FIG. 3. a) shows a typical Jahn-Teller distorted axial d⁹ spectrum with g_{∥}>g_{⊥} (g_{∥}=2.359, g_{⊥}=2.074), which is consistent with a ground state in which the unpaired electron resides in the d_{x2-y2} orbital.^{18,21,37,38} The EPR spectrum is distinctly different, however, from that due to [Cu(Indo)₂(Pyrro)₂] (g_{∥}=2.266, g_{⊥}=2.051) as a result of the different symmetries of the two complexes (FIG. 3, Table 5). There is no evidence of contamination with any appreciable amount of the dimer in the EPR spectrum obtained from a powdered sample of the monomeric complex, [Cu(Indo)₂(Py)₃]. In pyridine solutions, only signals due to mononuclear species were observed (g_{eff}=2.161), which shows that the dimeric structures were unstable in solutions containing an excess of pyridine. Crystals of mononuclear species could be obtained from these solutions, but they were slowly replaced with time by crystals of the less soluble dimer.

TABLE 5
X-band EPR Data for Cu(II)-Indo Complexes.

| | Dimeric resonance | | | | Monomeric resonance | | |
|---|---|---|---|---|---|---|---|
| Compounds | State | g_{\|\|}^{a} | g_{\|\|}^{b} | g_{⊥} | g_{\|\|} | g_{⊥} | g_{eff}^{c} |
| [Cu₂(Indo)₄(DMA)₂] | solid | 26.45 | 1.147 | 1.454 | 2.334 | 2.070 | 2.158 |
| [Cu₂(Indo)₄(THF)₂] | solid | 25.512 | 1.147 | 1.449 | 2.350 | 2.071 | 2.164 |
| [Cu₂(Indo)₄(ACN)₂] | solid | 31.176 | 1.139 | 1.455 | 2.335 | 2.077 | 2.163 |
| [Cu(Indo)₂(Py)₃] | solid | | | | 2.359 | 2.074 | 2.169 |
| [Cu₂(Indo)₄(Py)₂] | solution | | | | | 2.090 | 2.161 |
| [Cu(Indo)₂(Py)₃] | solution | | | | | 2.090 | 2.161 |
| [Cu(Indo)₂(Pyrro)₂] | solid | | | | 2.266 | 2.051 | 2.123 |

^{a}This corresponds to H_{z1} in FIG. 3.
^{b}This corresponds to H_{z2} in FIG. 3.
^{c}g_{eff} = ⅓(g|| + 2 g⊥), except solution-state taken from spectra.

### X-Ray Powder Diffraction

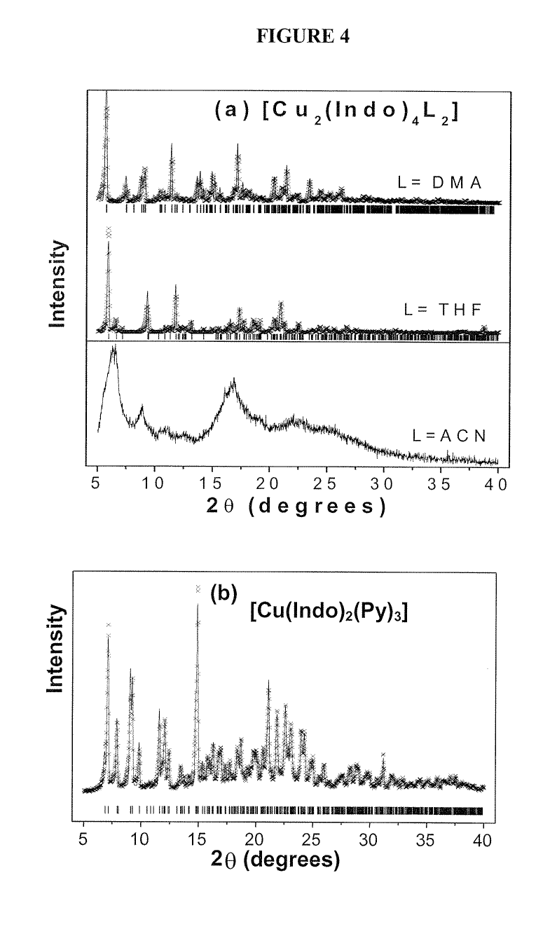

X-ray powder diffraction patterns for the dinuclear [Cu₂(Indo)₄L₂] (L=DMA, THF, or ACN) and the mononuclear [Cu(Indo)₂(Py)₃] complexes are shown in FIG. 4 and the short vertical marks show the positions of the Bragg reflections expected from the results of all the single-crystal analyses. The derived lattice parameters of [Cu₂(Indo)₄L₂] (L=DMA or THF) and [Cu(Indo)₂(Py)₃] are listed in Table 6, and the patterns can be used to distinguish between monomers and dimers.

TABLE 6
Lattice Parameters and Selected Details of Refinements of the
Powder Diffraction Patterns of [Cu₂(Indo)₄L₂] (L = DMA, THF) and
[Cu(Indo)₂(Py)₃] Complexes

| Param- | | | |
|---|---|---|---|
| eters | | | |
| Space | [Cu₂(Indo)₄(DMA)₂] | [Cu₂(Indo)₄(THF)₂] | [Cu(Indo)₂(Py)₃] |
| group | P 1̅ (#2) | P 1̅ (#2) | P2₁/c(#14) |
| a (Å) | 11.280(2) | 14.493(6) | 13.136(7) |
| b (Å) | 13.281(6) | 16.896(5) | 44.682(2) |
| c (Å) | 16.487(6) | 10.046(8) | 9.929(1) |
| α (°) | 100.17(2) | 106.58(2) | |
| β (°) | 100.61(5) | 89.97(0) | 94.10(5) |
| γ (°) | 110.94(9) | 109.95(2) | |

### Crystal and Molecular Structures

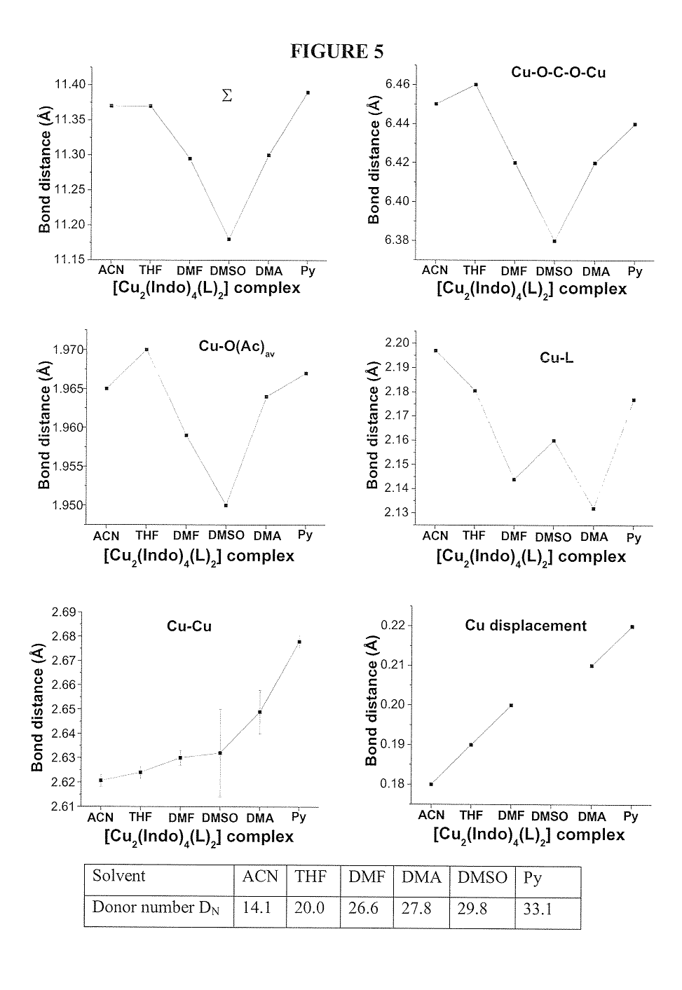

FIG. 5 shows a comparison of the bond distance and Cu displacement in the complexes [Cu₂(Indo)₄L₂] where L=ACN, THF, DMF, DMA, DMSO or Py. There are no systematic differences in the bonding parameters of these complexes with O-donors compared with complexes with the N-donor ligands, Py and ACN, except that the Cu—Cu bond was somewhat longer in the Py complex. FIG. 5 summarises the bond distance and Cu displacement from plane for dinuclear [Cu₂(Indo)₄L₂]. There are also no clear trends that distinguish the core geometry of complexes with stronger donor capacity ligands from those with weak donor capacity as the ternary ligand, except there are clear trends in the Cu—Cu distance and the Cu displacement from plane that reflects the donor capacity of the axial ligand, i.e., the Cu—Cu bond weakens as the donor strength of the solvent increases. Thus FIG. 5 demonstrates that increasing the donor strength favours monomer formation over dimer formulation and this has important implications in the preparation of monomeric complexes.

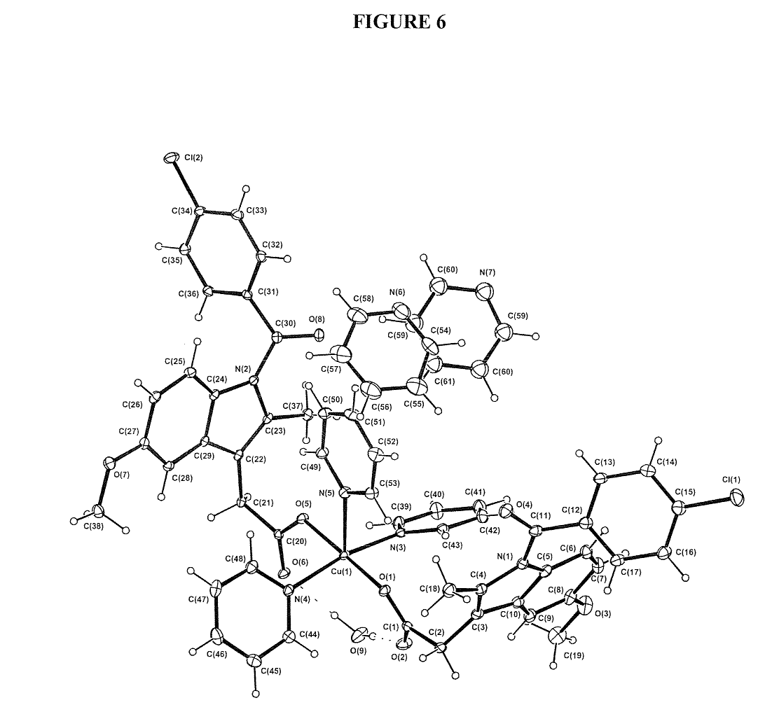

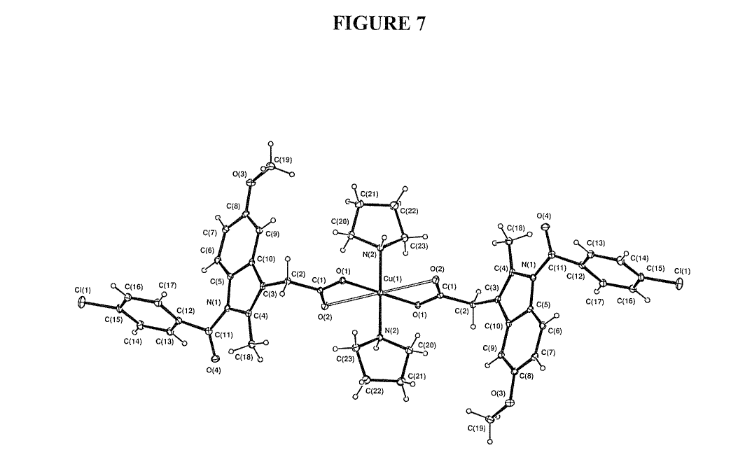

Crystal structure data for [Cu(Indo)₂(Py)₃] were collected at both 150(2) K and 295(2) K. Selected bond lengths and angles for [Cu(Indo)₂(Py)₃] at both temperatures and for [Cu(Indo)₂(Pyrro)₂] are given in Tables 7 and 8. The ORTEP³⁹ depictions of Complex 1 (with only one orientation of the disordered pyridine molecule shown) and Complex 2 are provided in FIGS. 6 and 7.

For mononuclear Complex 1, the carboxylate group of Indo is bound as a monodentate ligand and the structure is comprised of a five-coordinate Cu(II) centre with three monodentate pyridine ligands, similar to that reported for another monodentate Cu(II) carboxylate complex that contains the pyridine ligand and having the CuN₃O₂ chromophore.⁴¹ Complex 1 is an essentially five-coordinate square pyramidal Cu centre with the in-plane angular distortion away from the regular square-based pyramidal geometry and with a elongated apical Cu(1)-N(5) bond length of 2.317(2) Å. The two nitrogen atoms N(3) and N(4) and the carboxylate oxygen atoms O(1) and O(5) occupy trans positions in the basal plane with basal bond lengths of Cu—N 2.073(2), 2.067 (2) Å and Cu—O 1.9635(16), 1.9492 (16) Å. The N(3)-Cu(1)-N(4) angle of the basal plane is 166.21(8)°, while the O(5)-Cu(1)-O(1) angle is close to linear, 176.57(7)°.

Complex 2 may be described as a tetragonally distorted octahedron, with a four-coordinate square-planar bonding with weak off axis secondary coordination from the second ‘carbonyl’ oxygen of the carboxylate, which is bound as an unsymmetric bidentate ligand. The mononuclear Cu bonded in a trans square-planar arrangement to two pyrrolidine nitrogen atoms at Cu—N 2.051(2) Å and one short carboxylate oxygen atoms from each of two Indo ligands at Cu—O(1) 1.9719 (14) Å. The remote carboxylate oxygen atoms bind to the Cu atoms Cu . . . O(2)=2.5696(16) Å showing weak interactions. The O(1)-Cu(1)-N(2) angle is 93.22(7)°. This structure is comparable to those observed in the X-ray structures of mononuclear Cu(II) carboxylate complexes with the trans square-planar CuN₂O₂ . . . O₂ chromophore,^{21,38,42,43} such as Cu complexes of anti-inflammatory and anti-convulsant drugs, [Cu(aspirinate)₂(Py)₂].³⁸ and [Cu(niflumato)₂(3-PyMe)₂].⁴³

TABLE 7
Selected bond lengths (Å) and bond angles (°) of Complex 1.

| Bond lengths | 295(2) K | 150(2) K | Bond angles | 295(2) K | 150(2) K |
|---|---|---|---|---|---|
| Cu(1)—O(5) | 1.917(4) | 1.9492(16) | O(5)—Cu(1)—O(1) | 176.42(18) | 176.57(7) |
| Cu(1)—O(1) | 1.928(4) | 1.9635(16) | O(5)—Cu(1)—N(3) | 91.55(18) | 91.74(8) |
| Cu(1)—N(3) | 2.044(5) | 2.073(2) | O(1)—Cu(1)—N(3) | 91.29(18) | 91.01(7) |
| Cu(1)—N(4) | 2.047(5) | 2.067(2) | O(5)—Cu(1)—N(4) | 89.31(18) | 89.34(8) |
| Cu(1)—N(5) | 2.306(5) | 2.317(2) | O(1)—Cu(1)—N(4) | 88.41(18) | 88.48(8) |
| O(1)—C(1) | 1.289(7) | 1.294(3) | N(3)—Cu(1)—N(4) | 167.0(2) | 166.21(8) |
| O(2)—C(1) | 1.226(7) | 1.233(3) | O(5)—Cu(1)—N(5) | 86.77(18) | 86.89(7) |
| O(5)—C(20) | 1.265(6) | 1.287(3) | O(1)—Cu(1)—N(5) | 90.81(19) | 90.82(7) |
| O(6)—C(20) | 1.240(6) | 1.249(3) | N(3)—Cu(1)—N(5) | 95.04(19) | 95.17(8) |
| C(1)—C(2) | 1.527(8) | 1.541(3) | N(4)—Cu(1)—N(5) | 98.0(2) | 98.61(8) |
| C(20)—C(21) | 1.531(7) | 1.531(3) | C(1)—O(1)—Cu(1) | 122.6(4) | 121.03(16) |
| N(3)—C(39) | 1.323(7) | 1.349(3) | C(20)—O(5)—Cu(1) | 126.9(4) | 125.39(15) |
| N(3)—C(43) | 1.351(7) | 1.355(3) | C(39)—N(3)—Cu(1) | 124.1(5) | 122.52(17) |
| N(4)—C(44) | 1.342(7) | 1.345(3) | C(43)—N(3)—Cu(1) | 120.2(4) | 120.35(17) |
| N(4)—C(48) | 1.342(7) | 1.350(3) | C(39)—N(3)—C(43) | 115.7(6) | 117.1(2) |
| N(5)—C(53) | 1.336(7) | 1.353(3) | C(44)—N(4)—Cu(1) | 122.9(5) | 122.08(18) |
| N(5)—C(49) | 1.346(7) | 1.355(3) | C(48)—N(4)—Cu(1) | 121.3(5) | 120.80(18) |
| | | | C(44)—N(4)—C(48) | 115.5(6) | 116.8(2) |
| | | | C(53)—N(5)—Cu(1) | 121.8(5) | 121.15(17) |
| | | | C(49)—N(5)—Cu(1) | 122.2(4) | 121.37(17) |
| | | | C(53)—N(5)—C(49) | 115.9(6) | 117.3(2) |
| | | | O(2)—C(1)—O(1) | 124.7(6) | 125.6(2) |
| | | | O(6)—C(20)—O(5) | 125.4(5) | 125.2(2) |
| | | | O(2)—C(1)—C(2) | 120.3(6) | 119.9(2) |
| | | | O(1)—C(1)—C(2) | 115.0(6) | 114.5(2) |
| | | | O(6)—(20)—C(21) | 117.5(5) | 118.8(2) |
| | | | O(5)—C(20)—C(21) | 117.0(5) | 116.1(2) |
| | | | τ | 0.1570 | 0.1727 |

*Symmetry operation: (1) x, y, z; (2) −x, y + ½, −z + ½; (3) −x, −y, −z; (4) x, −y − ½, z − ½

TABLE 8
Selected bond lengths (Å) and bond angles (°) within Complex 2.

| | |
|---|---|
| Bond lengths (Å) | Bond angles (°) |

| | | | |
|---|---|---|---|
| Cu(1)—O(1) | 1.9719(14) | O(1)—Cu(1)—O(1)* | 180.0 |
| Cu(1)—O(2) | 2.5696(16) | O(1)—Cu(1)—N(2) | 93.22(7) |
| Cu(1)—N(2) | 2.051(2) | O(1)—Cu(1)—N(2) | 86.78(7) |
| O(1)—C(1) | 1.291(2) | N(2)—Cu(1)—N(2)* | 180.0 |
| O(2)—C(1) | 1.232(2) | C(1)—O(1)—Cu(1) | 103.37(13) |
| C(1)—C(2) | 1.528(3) | O(2)—C(1)—O(1) | 122.6(2) |
| N(2)—C(23) | 1.488(3) | O(2)—C(1)—C(2) | 122.27(19) |
| N(2)—C(20) | 1.493(3) | O(1)—C(1)—C(2) | 115.17(18 |

*Symmetry operation: (1) x, y, z (2) −x, −y, −z

### Discussion

### Synthesis

The donor strength of the solvent and the presence or absence of strong donor ligands in the solvent play an integral role in determining the nature of the coordination complexes containing carboxylate donors.^{3,20,21,41} Both monomer and dimer Cu complexes can be formed for a given ligand of the formula L¹; depending upon the electronic properties of the solvent or ligands present in the solvent, as evident by the results reported here where complexes were formed with a bidentate carboxylate bridged Cu-Indo dimer, monodentate bis(carboxylato) Cu-Indo monomer (Complex 1) and unsymmetrical bis(bidentate) chelates in monomers such as Complex 2. The axial ligands can be exchanged with the solvent used for the recrystallisation procedure, or strong donor ligands present in the solvent used for the recrystallisation procedure, and this leads to changes in Cu coordination, such as observed in the preparations of Complex 1 and Complex 2 and further examples in the literature.^{29,40,41} This is very important for designing pharmaceutical formulations since the solvents or the excipients used sometimes could lead to a change in the structure of Cu-Indo so potentially affecting biological activity, e.g., toxicity.

In the synthesis of Cu-Indo complexes, the preference for monomer over dimer formation in Cu-Indo complexes correlates with the donor capacity of the axial ligands, with strong donors such as Py and Pyrro, preferring monomers.

### UV-Vis Spectroscopy

There is debate in the literature^{2,28} as to whether the ε value of band I for some mononuclear and dinuclear Cu(II) complexes NSAIDs are similar. It was pointed out that the molar absoiptivities for the monomeric pyridine analogues of the Cu(II) complexes of the NSAIDs naprosyn, ε_{dmf}=301 M⁻¹ cm⁻¹, and ibuprofen, ε_{dmf}=263 M⁻¹ cm⁻¹,^{18,21} are similar to that of a dimeric DMSO Cu(II) complex of ibuprofen, ε_{dmf}=398 M⁻¹ cm⁻¹,^{18,28} Elsewhere, it has been pointed out that the value of the molar absorptivity for the dimeric DMSO Cu(II) complex of ibuprofen (ε=178 M⁻¹ cm⁻¹)^{25,28} is approximately half that for other dimeric Cu complexes. For the monomeric pyridine analogues of the Cu(II) complexes of the NSAIDs, naprosyn and ibuprofen, the solvent used to record the solution state UV-Vis spectra was DMF, which is different from the solvent ligand, pyridine. Obviously, ligand-exchange reactions can occur with the solvent that could lead to structural changes, which would be reflected in the UV-Vis spectra.¹⁸ Values have been repoited for the molar absorptivity for the dimeric DMSO Cu(II) complexes of naprosyn (ε_{DMSO}=457 M⁻¹ cm⁻¹) and ibuprofen, ε_{DMSO}=(380 M⁻¹ cm⁻¹) in DMSO as the solvent and the monomeric pyridine Cu(II) complexes of naprosyn (ε_{Py}=85 M⁻¹ cm⁻¹) and ibuprofen, (ε_{Py}=66 M⁻¹ cm⁻¹)⁸ in Py as the solvent. There is conflict in the ε values reported in 1990²⁵ and 1992¹⁸ papers with the same author for the dimeric DMSO Cu(II) complex of ibuprofen, which was later reported as 380 M⁻¹ cm⁻¹. Overall, it is clear that the intensities, e values, of band I in the solution-state UV-Vis spectra are much higher for dinuclear carboxylate complexes than for mononuclear complexes, which can be used to determine the presence of monomeric or dimeric units. Moreover, the position of band I can be used to distinguish between the five-coordinate complexes with monodentate ligands (e.g., Complex 1) and tetragonally distorted octahedral complexes containing unsymfetric chelating Indo ligands, such as Complex 2.

### Vibrational Spectra

Characterisation of [Cu₂(Indo)₄L₂] (L=DMA, ACN, THF), [Cu(Indo)₂(Py)₃] and [Cu(Indo)₂(Pyrro)₂] using solid-state FT IR spectroscopy also allowed the coordination mode of the carboxylate ligands to be distinguished from the shifts in the bands due to the v_{asym}C( [symbol] O)₂ stretches and the loss of the bands due to the v_{carbo}(O—H) stretches of IndoH. The shift in wavenumbers of the v_{sym}C( [symbol] O)₂ bands is different between dimeric bridging and monomeric unidentate coordination. All of the Δv values are ca. 200-220 cm⁻¹ for bridging [Cu₂(Indo)₄(L)₂], which are greater than for those for unidentate coordination in [Cu(Indo)₂(Py)₃] (178 cm⁻¹), which is consistent with reports in the literature on related complexes.^{2,21} However, the v_{sym}C( [symbol] O)₂ stretches that occur at lower frequencies of around 1400 cm⁻¹ and 1440 cm⁻¹ in the dimeric and monomeric complexes, respectively, are in the IR fingerprint region, where they overlap with other bands from the Indo ligand, the solvent ligand and uncoordinated solvent molecules of crystallisation, which make correct assignment of v_{sym}C( [symbol] O)₂ difficult and less certain. For example, the stretching vibrations for the pyridine ring (vC [symbol] C and vC [symbol] N) occur in the region between 1600-1430 cm⁻¹, which could overlap with the v_{sym}C( [symbol] O)₂ band at 1445 cm⁻¹. Solid-state IR spectra of the complexes were useful in revealing the presence of solvent stretching vibrations, such as those of DMA (v_{amide}(C═O) at 1654 cm⁻¹) and ACN (v_{C≡N} at 2363, 2335 and 2277 cm⁻¹), but this does not show that the solvent molecules are coordinated to the Cu centre.

### EPR Spectroscopy

The EPR spectra of the Indo complexes are diagnostic for distinguishing between monomers and dimers in both solution and the solid state.^{2,21,23,29,38} The results reported here also show the value of the EPR spectroscopy in determining the structure of the monomers, as distinctively different EPR spectra are obtained from the Py and Pyrro complexes due to their different symmetries.

### X-Ray Powder Diffraction

Examination of these patterns shows very distinct differences between monomer and dimer structures, which are again diagnostic. They also show that the bulk material is the same as that used to determine the single-crystal structure.

### Structural Trends

It is uncommon to have present a series of dimeric Cu(II) complexes with the same carboxylate bridging ligand where the apical ligand is changed over a range of the O- and N-donor capacities. This range also provides the first illustration of a comparison of the effects of axial ligands in dimeric Cu complexes with the relatively weak O- and N-donor capacity ligands, THF and ACN, and strong O- and N-donor ligands. The strength of donor capacity (acceptor number) is as follows:^{16,17}
ACN<THF<DMF<DMA<Py

Although the donor number of Pyrro does not appear to have been reported, it is also expected to be a strong donor ligand by analogy with other similar N-donor ligands.

There are clear trends in the Cu—Cu distance and the Cu displacement from plane that reflects the donor capacity of the axial ligand.¹ The weakening of the Cu—Cu bond with increasing donor capacity of the solvent explains why monomers are formed with N-donor solvents that are strong donor solvents and it is likely that these solvents in general will result in such complexes.

The carboxylate groups of both of the mononuclear complexes, Complex 1 and Complex 2, reveal the correlation⁴⁶ of an increase in the length of the bound carboxylate arm C—O(1), which is accompanied by a decrease in the length of the unbound or weakly bound arm C—O(2). Compared to dinuclear Cu-Indo complexes, there are no significant differences in the Cu—O(Ac) bond length, however, the C—O(Ac)ₐᵥ bond length in the dinuclear Cu-Indo complexes are shorter than the bound carboxylate arm C—O(1) and somewhat longer than the unbound or weakly bound arm C—O(2) in both mononuclear Cu-Indo complexes, Complex 1 and Complex 2. This is a consequence of delocalisation between the two C—O bonds in the dinuclear Cu-Indo complexes. Such differences account for the different shifts observed in the carboxylate stretching frequencies between the mononuclear and the dinuclear Cu-Indo complexes in their IR spectra.

Importantly the carboxylate electron delocalisation stabilised the dinuclear bridged Cu-Indo complexes compared with monodentate binding in [Cu(Indo)₂(Py)₃] (the Indo ligand was much more weakly bound in [Cu(Indo)₂(Py)₃]). Although the mononuclear complex, [Cu(Indo)₂(Pyrro)₂], exhibited only weak off axis secondary coordination from the second ‘carbonyl’ oxygen of the carboxylate, these two weak Cu . . . O(2) interactions exert a significant and crucial effect on the stabilisation of this complex to ligand substitution, which is reflected in the gastrointestinal toxicity studies (Example 2).

### Example 2 Efficacy and Safety in Rats: A Comparison of Different Pharmaceutical Formulations

This example compares the efficacy and safety of a complex of formula (1), bis(η²-O,O′-Indo)bis(pyrrolidine)copper(II), [Cu(Indo)₂(Pyrro)₂] (Complex 2), the dimer complex, [Cu₂(Indo)₄(DMF)₂], and the monomer [Cu(Indo)₂(Py)₃] (Complex 1) in a series of in vivo studies for the assessment of the complexes as anti-inflammatory agents and for their ability to induce acute gastrointestinal ulceration.

### Animals

Sprague-Dawley rats weighing 200-250 g were housed in metabolic cages four days before study and allowed free access to standard laboratory rat chow (Purina Rat Chow, Ralston Purina, St Louis Mo., USA) and tap water. Animals were supplied by the laboratory animal services at the University of Sydney and housed in the Bosch animal house facility of the University of Sydney at ambient temperature and humidity with a 12-h light-dark cycle. The experimental animal protocols were approved by the Animal Ethics Committee of the University of Sydney.

### Chemicals.

IndoH, carboxymethylcellulose (CMC) and carrageenan Type 1 were purchased from Sigma Aldrich. Technical grade formaldehyde was purchased from Ajax Chemicals (Auburn, Australia).

### Dosing Forms and Administration

Rats were orally dosed via a curved feeding needle (Harvard Apparatus) attached to a 1-mL syringe. IndoH or an equimolar indomethacin dose of the test compound ([Cu₂(Indo)₄(DMF)₂], [Cu(Indo)₂(Py)₃] or [Cu(Indo)₂(Pyrro)₂1] suspended in 0.5 mL of 2% (w/v) CMC solution were used in the treatments. The dose of each compound is listed in Table 9.

TABLE 9
Dose of each compound in the animal tests

| | Equimolar Indo |
|---|---|
| Compound | Dose (mg/kg) |
| IndoH | 10.00 |
| [Cu₂(Indo)₄(DMF)₂] | 11.90 |
| [Cu(Indo)₂(Py)₃] | 14.17 |
| [Cu(Indo)₂(Pyrro)₂] | 12.86 |

### Inhibition of Carrageenan-Induced Paw Edema (Anti-Inflammatory Activity of the Test Compounds)

The control cohort was dosed solely with CMC (2%) solution. Inflammation was induced one hour after dosing with the NSAID (or vehicle), by injecting with carrageenan (0.1 mL, 2% w/v in isotonic saline) into the plantar region of the hind paw (n=3) (Winter, C. A.; Flataker, L., Pharmacol. Exp. Ther. 1965, 150, 165-171). The thickness of the paw was measured at the ventral dorsal footpad using digital callipers prior to dosing and at 3 and 5 h after carrageenan injection. Paw volume was measured prior to dosing and at 3 and 5 h after carrageenan injection by submerging the right hind paw in water up to an ink mark on the skin over the lateral malleus.⁴ The vessel containing the water was tared to zero on a top pan balance and the volume of fluid displaced was measured directly as a positive force (in grams). As the density of water is 1 g mL⁻¹, a measurement of 1 g corresponds to a volume of 1 mL. The mean percent edema or percent inhibition of edema was determined as:

$$\begin{matrix}\% edema = [ \frac{\begin{matrix}volume of inflamed paw - \\ volume of paw prior to dosing\end{matrix}}{volume of paw prior to dosing} ] \times 100 & I \\ \% inhibition = [ \frac{\% edema ( control ) - \% edema ( drug )}{\% edema ( control )} ] \times 100 & II\end{matrix}$$

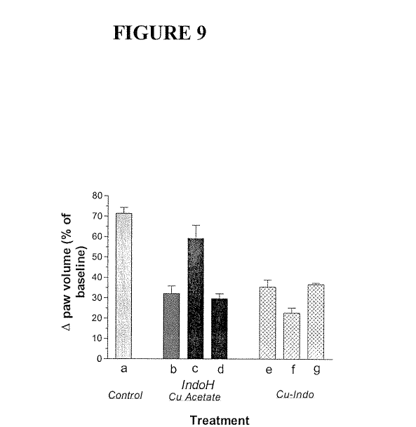

The results are shown in FIG. 9.

### Acute Macroscopic Gastric Damage.

### Method 1

Rats were fasted with access to water for 24 h prior to dosing and 3 hours post dosing. Each group of rats (n=3-6 per group) was orally dosed with IndoH, or an equimolar IndoH dose of test compound listed in Table 9, or vehicle. Three hours after administration of the test compound, the rats were euthanased and the stomach was excised and opened by incision along the greater curvature. The stomach was rinsed, submerged in 10% formaldehyde for 1 h and the extent of macroscopic gastric toxicity was examined, which is expressed as the summation of the area of macroscopic ulcerations (mm²).

### Method 2

After the aforementioned anti-inflammatory activity experiments, the rats were immediately euthanased. Similarly, the stomach was excised and opened by incision along the greater curvature for the examination of the macroscopic ulcerations (mm²).

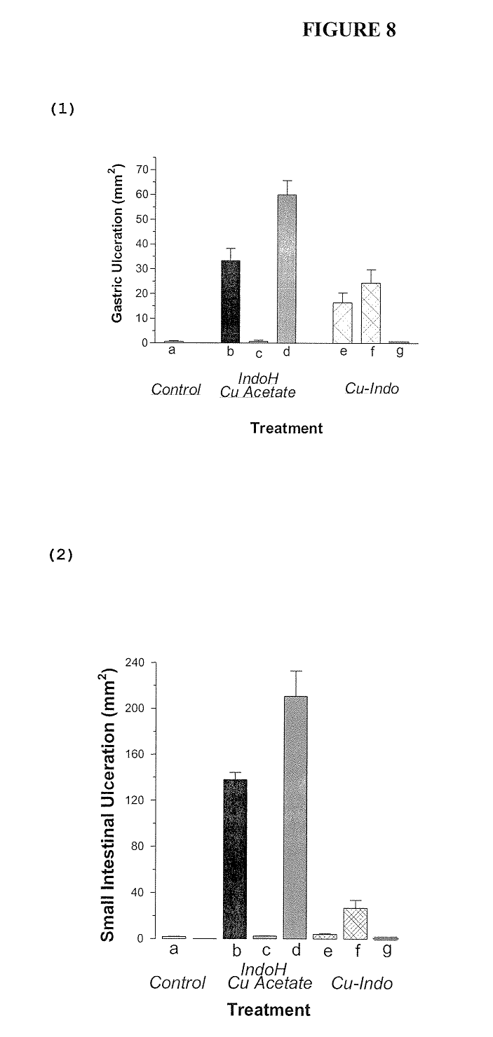

Since there were no significant differences in the results obtained by the two methods, the results shown in FIG. 8(1) are those of the two methods combined.

### Small Intestinal Macroscopic Damage

Rats were allowed free access to food and water throughout and prior to the assay period. Each group of rats (n=3-6 per group) was orally dosed with IndoH, or an equimolar IndoH dose of test compound listed in Table 9, or vehicle. At 24 h after dosing, rats were euthanased and the entire small intestine was excised and flushed with water to expel the intestinal contents and opened along the anti-mesenteric side. The intestine was examined from 10 cm distal to the ligament of Treitz to the ileocecal junction for macroscopic ulcerations. The degree of ulcerations is expressed as the summation of the area of macroscopic ulcerations (mm²).

### Statistical Analysis

All inhibition of carrageenan-induced paw edema and gastrointestinal ulceration data are expressed as the standard error of the mean (±sem). Comparisons among the control and treatment groups were made using one-way analysis of variance followed by a Student-Newman-Keuls t-test using the GraphPad Instat statistical program. With all analyses, an associated probability (P value) of less than 5% (P<0.05) was considered significant. The calculation of the power of the experiment to compare two treatment groups with a P-value threshold of 0.05 was determined using the GraphPad StatMate program (GraphPad Instat; version 3.01 for WIN95/NT, GraphPad Software Inc., 1998).

### Results

### Acute Macroscopic Gastric Damage

FIG. 8(1) shows the results of the macroscopic gastric ulcerations induced by IndoH and equimolar Indo dose of test compounds in 2% (w/v) CMC solution. [Cu₂(Indo)₄(DMF)₂] and [Cu(Indo)₂(Pyrro)₂] show significant reductions (P<0.01) in gastric ulcerations as compared to those induced by IndoH and a physical mixture of IndoH and Cu-acetate. [Cu(Indo)₂(Pyrro)₂] also exhibited a significant reduction in gastric ulceration compared with [Cu₂(Indo)₄(DMF)₂]. There is no significant difference in the gastric ulcerations induced by [Cu(Indo)₂(Py)₃] or IndoH.

Interestingly, gastric damage was significantly increased in rats treated with a physical mixture of IndoH and Cu-acetate compared with rats treated with the IndoH alone. Similar trends were observed in the small intestine ulcerations (FIG. 8(2)).

In a second experiment, using a different sample of [Cu(Indo)₂(Pyrro)₂] that was precipitated from solution with diethyl ether, the average gastric ulceration for four rats was higher at 21 mm², which is the same, within experimental error, as was observed for [Cu₂(Indo)₄(DMF)₂] at the same dose of Indo. The efficacy was also similar to [Cu₂(Indo)₄(DMF)₂].

### Small Intestinal Ulceration

The results (FIG. 8(2)) show that [Cu(Indo)₂(Pyrro)₂] has a similar low small intestinal toxicity as is observed for [Cu₂(Indo)₄(DMF)₂], but [Cu(Indo)₂(Py)₃] has a significantly higher toxicity (FIG. 8(2)).

### Efficacy of the New Complexes

FIG. 9 shows that Complex 1 is as effective as [Cu₂(Indo)₄(DMF)₂] in reducing inflammation.

### Discussion

The results establish that the mononuclear complex of formula (1) has comparable efficacy as the Cu-Indo dimers currently used in veterinary applications, and surprisingly causes similar or less ulceration in the stomach and somewhat less ulceration in the small intestine than the dimer. In contrast, the monomer of the formula (2) caused more ulceration in the stomach and small intestine than the dimer.

### Example 3 Preparation and Characterisation of Other Mononuclear Complexes

### A Preparation of bis(η²-O,O′-Indo)bis(imidazole)copper(II) ([Cu(Indo)₂(Im)₂])

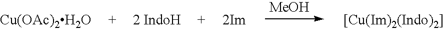

(a): Cu(OAc)₂H₂O (54 mg, 0.2704 mmol) in methanol (4 mL) and water (6 drops) was sonicated for 0.5 hour to dissolve the Cu complex. Indomethacin (200 mg, 0.5590 mmol) and imidazole (38 mg, 0.5581 mmol) in methanol (6 mL) were stirred until the solid dissolved. The solution of Cu²⁺ was added drop-wise to the solution of indomethacin and imidazole at ˜30° C. with stirring. The mixture was stirred for 15 minutes and the solution changed colour from green/blue to dark blue. Solid was formed after 5 minutes and filtration to yielded the title compound as a blue/purple solid. The solid was washed with methanol (2 mL) once, and dried under nitrogen flow. C₄₄H₃₆Cl₂CuN₆O₈=911.25, Calcd.: C, 58%, H, 3.98%, N, 9.22%, Cl, 7.78%, Cu, 6.97%; Found: C, 57.79%, H, 4.37%, N, 8.89%, Cl, 7.83%, Cu, 6.67%.

(b): Cu(OAc)₂.H₂O (76.2 mg, 0.3817 mmol) in methanol (8 mL) and water (4 drops) was sonicated for 0.5 hour to dissolve the Cu complex. Indomethacin (274 mg, 0.7633 mmol) and imidazole (54 mg, 0.7932 mmol) in methanol (8 mL) were stirred to dissolve the solid. The solution of Cu²⁺ was added drop-wise to the solution of indomethacin and imidazole at room temperature with stirring. The mixture was stirred for 3 minutes. The solution was dark blue and was set aside for crystallization. Two hours later, crystals were formed. The dark blue/purple crystals (291 mg, 83.7%) were separated by decanting the solvent and were washed with methanol (3 mL) once, and dried under nitrogen flow. C₄₄H₃₆Cl₂CuN₆O₈=911.25, Calcd.: C, 58%, H, 3.98%, N, 9.22% Cl, 7.78%, Cu, 6.97%; Found: C, 57.16%, H, 4.21%, N, 8.76%, Cl, 7.88%, Cu 6.72%.

(c): Cu(OAc)₂.H₂O (0.2809 g, 1.407 mmol) in methanol (25 mL) and water (1 mL) was sonicated for 0.5 hour to dissolve the Cu complex. Indomethacin (1.0067 g, 2.814 mmol) and imidazole (0.1915 g, 2.814 mmol) in methanol (25 mL) were sonicated to dissolve the solids. The solution of Cu²⁺ was added drop-wise to the solution of indomethacin and imidazole at room temperature with stirring. A purple solid was formed. A little more imidazole was added to the mixture. The mixture was stirred for 2 minutes and the colour of the solution became more blue. The blue/purple solid was collected by filtration and was washed with 95% ethanol (5 mL) once.

The product of (a), (b) and (c) above was identified as [Cu(Indo)₂(Im)₂] from the IR spectra, UV-Vis spectra, EPR spectra and single crystal X-ray structure of the product.

A blue prism like crystal was attached with Exxon Paratone N, to a short length of fibre supported on a thin piece of copper wire inserted in a copper mounting pin. The crystal was quenched in a cold nitrogen gas stream from an Oxford Cryosystems Cryostream. An APEXII-FR591 diffractometer employing graphite monochromated MoKα radiation generated from a rotating anode was used for the data collection. Cell constants were obtained from a least squares refinement against 14152 reflections located between 5 and 60° 2θ. Data were collected at 150(2) K with ω+φ scans to 61° 2θ. The data integration and reduction were undertaken with SAINT and XPREP,⁷ and subsequent computations were carried out with the WinGX,⁹ and XTAL¹⁰ graphical user interfaces. An empirical absorption correction determined with SADABS⁴⁷ was applied to the data.

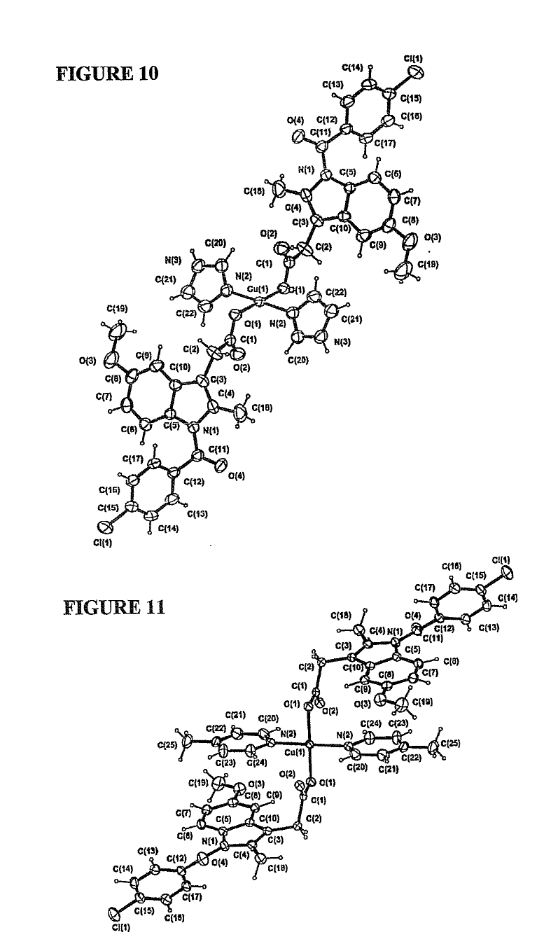

The structure was solved in the space group P21/n(#14) by direct methods with SHELXS-97,¹³ and extended and refined with SHELXL-97.¹³ The non-hydrogen atoms in the asymmetric unit were modelled with anisotropic displacement parameters, and in general a riding atom model was used for the hydrogen sites. An ORTEP³⁹ depiction of the molecule with 50% displacement ellipsoids is provided in FIG. 10.

### B Preparation of bis(η²-O,O′-Indo)bis(4-picoline)copper(II) ([Cu(Indo)₂(4-Pic)₂])

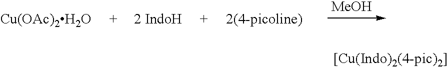

(a): Cu(OAc)₂—H₂O (65.6 mg, 0.3286 mmol) in methanol (10 mL) and water (4 drops) was sonicated for 0.5 hour to dissolve the Cu complex. Indomethacin (295 mg, 0.8245 mmol) and freshly distilled 4-picoline (77 mg, 0.8268 mmol) in methanol (8 mL) were sonicated to dissolve the solids. The solution of Cu²⁺ was added drop-wise to the solution of indomethacin and 4-picoline at ˜50° C. with stirring. The solution changed to a light green colour and solids were formed. The mixture was stirred for 15 minutes until the solid dissolved. More indomethacin and 4-picoline were added to the mixture solution. The solution was filtered and was set aside for crystallization. After half an hour, dark blue crystals were formed. The dark blue/purple crystals were separated by decanting the solvent, and washed with methanol (2×2 mL) and finally, dried under nitrogen flow.

C₅₀H₄₄Cl₂CuN₄O₈=963.37, Calcd.: C, 62.34%, H, 4.60%, N, 5.82%, Cl, 7.36%, Cu, 6.60%; Found: C, 62.25%, H, 4.72%, N, 5.80%, Cl, 7.55%, Cu, 6.40%.

(b): Cu(OAc)₂—H₂O (74.3 mg, 0.3721 mmol) in methanol (8 mL) and water (4 drops) was sonicated for 0.5 hour to dissolve the Cu complex. Indomethacin (266.3 mg, 0.7443 mmol) in methanol (6 mL) were sonicated the solid dissolved. Freshly distilled 4-picoline (69.3 mg, 0.7443 mmol) was added to the indomethacin solution. The solution of Cu²⁺ was added drop-wise to the solution of indomethacin and 4-picoline at room temperature with stirring. The solution changed to a dark green/blue colour. The mixture was stirred for 15 minutes and was then set aside for crystallization. After two hours, dark blue crystals were formed. The dark blue/purple crystals (0.3364 g, 93.8%) were separated by decanting the solvent, and washed with methanol (3 mL) and finally, dried under nitrogen flow. C₅₀H₄₄Cl₂CuN₄O₈=963.37, Calcd.: C, 62.34%, H, 4.60%, N, 5.82% Cl, 7.36%, Cu, 6.60%; Found: C, 62.24%, H, 4.60%, N, 5.82%, Cl, 7.39%, Cu, 6.42%.

(c): Cu(OAc)₂—H₂O (280.9 mg, 1.407 mmol) in methanol (29.5 mL) and water (0.5 mL) was sonicated for 0.5 hour to dissolve the Cu complex. Indomethacin (1.0004 g, 2.814 mmol) in methanol (30 mL) was sonicated until it dissolved. Freshly distilled 4-picoline (311 mg, 3.339 mmol) was added to the indomethacin solution. The solution of Cu²⁺ was added drop-wise to the solution of indomethacin and 4-picoline at room temperature with stirring. The solution changed to light green colour and solids were formed. More 4-picoline was added to the mixture solution until the solid was dissolved and the solution was dark blue colour. The solution was set aside for crystallization. After two hours, dark blue/purple crystals were formed. The dark blue/purple crystals were separated by decanting the solvent, and washed with methanol (2 mL). The crystals were not dried (after drying, their colour changed to grey).

The product of (a), (b) and (c) above was identified as [Cu(Indo)₂(4-pic)₂] from the IR spectra, UV-Vis spectra, EPR spectra and single crystal X-ray structure of the product.

A blue prism like crystal was attached with Exxon Paratone N, to a short length of fibre supported on a thin piece of copper wire inserted in a copper mounting pin. The crystal was quenched in a cold nitrogen gas stream from an Oxford Cryosystems Cryostream. A Bruker SMART 1000 CCD diffractometer employing graphite monochromated MoKα radiation generated from a sealed tube was used for the data collection. Cell constants were obtained from a least squares refinement against 8204 reflections located between 4.6 and 50.3° 2θ. Data were collected at 150(2) K with ω scans to 56.6° 2θ. The data integration and reduction were undertaken with SAINT and XPREP⁷ and subsequent computations were carried out with the WinGX⁹ and XTAL¹⁰ graphical user interfaces. The intensities of 184 standard reflections recollected at the end of the experiment did not change significantly during the data collection. A Gaussian absorption correction^{9,11} was applied to the data.

The structure was solved in the space group P 1̅(#2) by direct methods with SHELXS-97,¹³ and extended and refined with SHELXL-97.¹³ The non-hydrogen atoms in the asymmetric unit were modelled with anisotropic displacement parameters. A riding atom model with group displacement parameters was used for the hydrogen atoms. An ORTEP³⁹ depiction of the molecule with 50% displacement ellipsoids is provided in FIG. 11.

### Other Complexes.

Similar complexes with 3-pic and pyrazine as various other heterocylces were obtained using techniques as described above. They had similar spectroscopic properties and colours as the complexes characterised by X-ray crystallography.

### Results

The crystal data are summarised in Table 10, and bond length and angle data are summarised in Tables 11 and 12, respectively, for [Cu(Indo)₂(Im)₂] and [Cu(Indo)₂(4-pic)₂]. The complexes can be described as a square-planar complex with weak axial interactions with the second oxygen of the carboxylate ligands or a strongly tetragonally distorted octahedral complex with the equatorial sites being occupied by two nitrogens of the imidazole and one oxygen each from the two Indo ligands. The distortion for these complexes is somewhat larger than those in the structure of the pyrro complex.

TABLE 10
Crystal Data for [Cu(Indo)₂(Im)₂] and [Cu(Indo)₂(4-pic)₂]

| | [Cu(Indo)₂(Im)₂] | [Cu(Indo)₂(4-pic)₂] |
|---|---|---|
| Formula of the | C₄₄H₃₈Cl₂CuN₆O₈ | C₅₂H₅₂Cl₂CuN₄O₁₀ |
| Refinement Model | | |
| Model Molecular | 913.24 | 1027.42 |
| Weight | | |
| Crystal System | Monoclinic | Triclinic |
| Space Group | P21/n(#14) | P 1(#2) |
| A | 13.4807(14) Å | 10.3003(17) Å |
| B | 8.9756(9) Å | 10.9527(18) Å |
| C | 17.4675(18) Å | 12.118(2) Å |
| α | | 99.926(3)° |
| β | 97.173(6)° | 95.766(3)° |
| γ | | 113.254(3)° |
| V | 2097.0(4) Å³ | 1215.6(3) Å³ |
| D_{c} | 1.446 g cm⁻³ | 1.403 g cm⁻³ |
| Z | 2 | 1 |
| Crystal Size | 0.25 × 0.25 × 0.08 mm³ | 0.36 × 0.30 × 0.29 mm |
| Crystal Colour | blue | blue |
| Crystal Habit | prism | prism |
| Temperature | 150(2) K | 150(2) K |
| λ(MoKα) | 0.71073 Å | 0.71073 Å |
| μ(MoKα) | 0.710 mm⁻¹ | 0.623 mm⁻¹ |
| T(SADABS)_{min.max} | 0.919, 1.000 | 0.794, 0.870 |
| 2θₘₐₓ | 61.22° | 56.62° |
| hkl range | −19 19, −12 12, −24 24 | −13 13, −14 14, −16 16 |
| N | 42195 | 12275 |
| N_{ind} | 6405(R_{merge} 0.0307) | 5722(R_{merge} 0.0280) |
| N_{obs} | 5038(I > 2σ(I)) | 5147(I > 2σ(I)) |
| Nᵥₐᵣ | 283 | 318 |
| Residuals R1(F), | 0.0397, 0.1315^{1,2} | 0.0307, 0.0825^{1,3} |
| wR2(F²) | | |
| GoF(all) | 1.063 | 1.183 |
| Residual Extrema | −0.471, 0.614 e⁻ Å⁻³ | −0.317, 0.348 e⁻ Å⁻³ |

¹R1 = Σ||Fₒ| − |F_{c}||/Σ|Fₒ| for Fₒ > 2σ(Fₒ); wR2 = (Σw(Fₒ² − F_{c}²)²/Σ(wF_{c}²)²)^{1/2} all reflections
²w = 1/[σ²(Fₒ²) + (0.0810P)² + 0.3438P] where P = (Fₒ² + 2F_{c}²)/3
³w = 1/[σ²(Fₒ²) + (0.03P)² + 0.4P] where P = (Fₒ² + 2F_{c}²)/3

TABLE 11
Selected Bond Lengths (Å) and bond angles (°) within
[Cu(Indo)₂(Im)₂]

| Bond lengths (Å) | | Bond angles (°) | |
|---|---|---|---|
| Cu(1)—O(1) | 1.9447(11) | O(1)—Cu(1)—O(1) * | 180.00(6) |
| Cu(1)—O(2) | 2.937 | O(1)—Cu(1)—N(2) | 90.03(5) |
| Cu(1)—N(2) | 1.9960(15) | O(1)—Cu(1)—N(2) | 89.97(5) |
| O(1)—C(1) | 1.2813(19) | N(2)—Cu(1)—N(2)* | 180.00(8) |
| O(2)—C(1) | 1.235(2) | C(1)—O(1)—Cu(1) | 110.62(10) |
| C(1)—C(2) | 1.516(2) | O(2)—C(1)—O(1) | 124.33(14) |
| N(2)—C(22) | 1.373(2) | O(2)—C(1)—C(2) | 122.18(14) |
| N(2)—C(20) | 1.323(2) | O(1)—C(1)—C(2) | 113.48(14) |

TABLE 12
Selected Bond Lengths (Å) and bond angles (°) within
[Cu(Indo)₂(4-pic)₂]

| Bond lengths (Å) | | Bond angles (°) | |
|---|---|---|---|
| Cu(1)—O(1) | 1.9735(10) | O(1)—Cu(1)—O(1) * | 180.00(6) |
| Cu(1)—O(2) | 2.739 | O(1)—Cu(1)—N(2) | 88.28(5) |
| Cu(1)—N(2) | 2.0133(12) | O(1)—Cu(1)—N(2) | 91.72(5) |
| O(1)—C(1) | 1.2723(17) | N(2)—Cu(1)—N(2)* | 180.0 |
| O(2)—C(1) | 1.2522(18) | C(1)—O(1)—Cu(1) | 103.57(9) |
| C(1)—C(2) | 1.5232(19) | O(2)—C(1)—O(1) | 122.42(13) |
| N(2)—C(24) | 1.3377(19) | O(2)—C(1)—C(2) | 120.05(13) |
| N(2)—C(20) | 1.338(2) | O(1)—C(1)—C(2) | 117.49(12) |

### Example 4

### In Vivo Anti-Inflammatory Efficacy and Gastrointestinal Toxicity

### Experimental

These studies were conducted as described in Example 2, except that the monomer Cu complexes were thoroughly dispersed in an MCT paste by mechanical mixing of the complex with the paste. Some experiments were also performed with the complexes dispersed in 2% CMC.

### Results

The results are summarised in Table 13 for rats treated with the monomers dispersed in MCT paste at equivalent concentrations of Indo between 1 and 10 mg/kg). At 10 mg/kg Indo, the Cu monomer complexes exhibit similar efficacy as IndoH when mixed with MCT paste, and the small intestinal ulceration was reduced by a factor of two to three compared to IndoH and the gastric ulceration was also lower although not significantly so because of the large variations. Since the gastric ulceration was even higher (1 60±10 mm²) with a physical mixture of copper acetate and IndoH at the same molar concentrations, the monomers have a significant effect in reducing the gastrointestinal toxicity of IndoH, even at an order of magnitude higher than the therapeutic dose.

At 2 mg/kg Indo in MCT paste, the efficacy was just beginning to drop and a significant reduction was observed at 1 mg/kg. The extent of gastric ulceration at these lower concentrations is not significant and shows that the Cu complexes can be used safely at the therapeutic doses.

When the complexes were dispersed in 2% CMC at 2 mg/kg Indo, the efficacy was reduced considerably, and similar low levels of gastric toxicity were observed.

TABLE 13
Efficacy in the Rat Paw Oedema Assay and Gastrointestinal Toxicity
at the doses of 1-10 mg/kg (Indo equivalent) of the drug dispersed
in MCT paste and 2 mg/kg (Indo equivalent) in 2% CMC.

| | | | |
|---|---|---|---|
| | | | Small |
| | | | Intestine |
| | | Gastric | Ulceration |
| Drug used | Inhibition % | Ulceration | (mm²) |

10 mg/kg of Indo dispersed in MCT paste

| | | | |
|---|---|---|---|
| Control | 0 | 0 | 0 |
| IndoH | 54 ± 12 | 115 ± 30 | 80 ± 7 |
| [Cu(Im)₂(Indo)₂] | 45 ± 16% | 55 ± 42 | 42 ± 24 |
| [Cu(Indo)₂(4-pic)₂] | 57 ± 19% | 71 ± 42* | 28 ± 19 |

2 mg/kg of Indo dispersed in MCT paste

| | | |
|---|---|---|
| Control | 0 | 0 |
| [Cu(Im)₂(Indo)₂] | 39% | 3 ± 42 |
| [Cu(Indo)₂(4-pic)₂] | 54% | 1 ± 1 |

2 mg/kg of Indo in 2% CMC

| | | |
|---|---|---|
| Control | 0 | 0 |
| [Cu(Im)₂(Indo)₂] | 11% | 3 ± 3 |
| [Cu(Indo)₂(4-pic)₂] | 16% | 1 ± 1 |

1 mg/kg of Indo dispersed in MCT paste

| | | | |
|---|---|---|---|
| Control | 0 | 0 | 0 |
| [Cu(Im)₂(Indo)₂] | 31% | 2 ± 2 | |
| [Cu(Indo)₂(4-pic)₂] | 28% | 1 ± 1 | |

*faeces in the stomach of one rat.

### Discussion

Even though the monomer complexes are not as stable as the dimer, it is clear that when they are mixed with MCT paste, there is sufficient stability that they have an enhanced safety profile over Indo and the complexes are both safe and highly efficacious at 2 mg/kg. These complexes appear to be somewhat more GI toxic than the pyrro complex at the high concentration, which is consistent with the longer second axial bond to the Cu, making the Indo ligand less tightly held.

Dispersion of the solid into MCT paste also results in higher efficacy than when it is dispersed in 2% CMC, which indicates the MCT paste assists in the absorption of the drug.

Such complexes also have the ability to deliver N-heterocyclic ligands that are themselves active against a number of conditions.

Although the present invention has been described hereinbefore with reference to a number of preferred embodiments, it will be appreciated by persons skilled in the art that numerous variations and/or modifications may be made to the invention without departing from the spirit or scope of the invention as broadly described. The present embodiments are, therefore, to be considered in all respects as illustrative and not restrictive.

### REFERENCES

(1) Weser, U.; Sellinger, K. H.; Lengfelder, E.; Wemer, W.; Strahle, J. Biochim. Biophys. Acta 1980, 631, 232-245.
(2) Weder, J. E.; Hambley, T. W.; Kennedy, B. J.; Lay, P. A.; MacLachlan, D.; Bramley, R.; Delfs, C. D.; Murray, K. S.; Moubaraki, B.; Warwick, B.; Biffin, J. R.; Regtop, H. L. Inorg. Chem. 1999, 38, 1736-1744.
(3) Weder, J. E.; Dillon, C. T.; Hambley, T. W.; Kennedy, B. J.; Lay, P. A.; Biffin, J. R.; Regtop, H. L.; Davies, N. M. Coord. Chem. Rev. 2002, 232, 95-126.
(4) Fereidoni, M.; Ahmadiani, A.; Semnanian, S.; Javan, M. J. Pharmacol. Toxicol. Methods 2000, 43, 11-14.
(5) Zhou, Q.; Hambley, T. W.; Kennedy, B. J.; Lay, P. A.; Turner, P.; Warwick, B.; Biffin, J. R.; Regtop, H. L. Inorg. Chem. 2000, 39, 3742-3748.
(6) Figgis, B. N.; Lewis, J. In Modern Coordination Chemistry; Lewis, J.; Wilkins, R. G., Eds.; Interscience: New York, 1960; pp 400-454.
(7) Bruker SMART, SAINT, XPREP, Area detector control and data integration and reduction software; Bruker Analytical X-ray Instruments Inc., Madison, Wis., USA, 1995.
(8) Molecular Structure Corporation, TEXSAN for Windows: Single Crystal Structure Analysis Software, MSC, 3200 Research Forest Drive, The Woodlands, Tex. 77381, USA, 1997-1998.
(9) WinGX, Farrugia, L. J., J. Appl. Crystallogr. 1999, 32, 837-838.
(10) Hall, S. R.; du Boulay, D. J. & Olthof-Hazekamp, R., Xtal 3.6 System; University of Western Australia, 1999.
(11) Coppens, P.; Leiserowitz, L.; Rabinovich, D. Acta Ciystallogr. 1965, 18, 1035-1038.
(12) Altomare, A.; Cascarano, G.; Giacovazzo, C.; Guagliardi, A. J. Appl. Crystallogr. 1993, 26, 343-350.
(13) (a) Sheldrick, SHELXS-97, Program for Crystal Structure Refinement, 1997;
(b) SHELXS-97, Program for Crystal Structure Solution;
(c) SHELXH-97, Program for Crystal Structure Refinement, University of Göttingen, Göttingen, Germany.
(14) Altomare, A.; Burla, M. C.; Camalli, M.; Cascarano, G. L.; Giacovazzo, C.; Guagliardi, A.; Moliterni, A. G. G.; Polidori, G.; Spagna, R. J. Appl. Crystallogr. 1999, 32, 115-119.
(15) Sheldrick, G. M., SHELX97 Programs for Crystal Structure Analysis; University of Göttingen, Institüt für Anorganische Chemie der Universität, Tammanstrasse 4, D-3400 Gottingen, Germany, 1998.
(16) Fawcett, R. W. J. Phys. Chem. 1993, 97, 9540-9546.
(17) Gutmann, V. Coord. Chem. Rev. 1976, 18, 225-255.
(18) Dendrinou-Samara, C.; Jannakoudakis, P. D.; Kessissoglou, D. P.; Manoussakis, G. E.; Mentzafos, D.; Terzis, A. J. Chem. Soc. Dalton Trans. 1992, 3259-3264.
(19) Abuhijleh, A. L.; Woods, C.; Ahmed, I. Y. Inorg. Chim. Acta 1991, 190, 11-17.
(20) Abuhijleh, A. L.; Woods, C.; Bogas, E.; Le Guenniou, G. Inorg. Chim. Acta 1992, 195, 67-71.
(21) Abuhijleh, A. L.; Woods, C. Inorg. Chim. Acta 1993, 209, 187-193.
(22) Abuhijleh, A. L. J. Inorg. Biochem. 1994, 55, 255-262.
(23) Viossat, B.; Daran, J.-C.; Savouret, G.; Morgant, G.; Greenaway, F. T.; Dung, N.-H.; Pham-Tran, V. A.; Sorenson, J. R. J. J. Inorg. Biochem. 2003, 96, 375-385.
(24) Kögerler, P.; Williams, P. A. M.; Parajòn-Costa, B. S.; Baran, E. J.; Lezama, L.; Rojo, T.; Müller, A. Inorg. Chim. Acta 1998, 268, 239-248.
(25) Dendrinou-Samara, C.; Kessissoglou, D. P.; Manoussakis, G. E.; Mentzafos, D.; Terzis, A. J. Chem. Soc. Dalton Trans. 1990, 959-965.
(26) Melnik, M.; Poto\check{c}\check{n}ak, I.; Macà\check{s}kovà, L.; Miklo\check{s}, D.; Holloway, C. E. Polyhedron 1996, 15, 2159-2164.
(27) Catterick, J.; Thornton, P. Adv. Inorg. Chem. Radiochem. 1977, 20, 291-362.
(28) Weder, J. E., Thesis: Characterisation of Copper(II) Dinuclear Complexes of the Non-Steroidal Anti-Inflammatory Drug Indomethacin; The University of Sydney, Sydney, 2000.
(29) Agterberg, F. P. W.; Provò Kluit, H. A. J.; Driessen, W. L.; Reedijk, J.; Oevering, J.; Buijs, W.; Veldman, N.; Lakin, M. T.; Spek, A. L. Inorg. Chim. Acta 1998, 267, 183-192.
(30) Hadjikostas, C. C.; Katsoulos, G. A.; Sigalas, M. P.; Tsipis, C. A.; Mrozinski, J. Inorg. Chim. Acta 1990, 167, 165-169.
(31) Figgis, B. N.; Martin, R. L. J. Chem. Soc. 1956, 3837-3846.
(32) Melnik, M. Coord. Chem. Rev. 1981, 36, 1-44.
(33) Kokot, E.; Martin, R. L. Inorg. Chem. 1964, 3, 1306-1312.
(34) Casanova, J.; Alzuet, G.; Latorre, J.; Borràs, J. Inorg. Chem. 1997, 36, 2052-2058.
(35) Doedens, R. J. Prog. Inorg. Chem. 1976, 21, 209-231.
(36) Ahmed, I. Y.; Abu-hijleh, A. L. Inorg. Chim. Acta 1982, 61, 241-246.
(37) Hathaway, B. J.; Editor-In-Chief, S. G. W., Executive Eds. R. D. Gillard, J. A McCleverty, Ed.; Pergamon Press: Oxford, 1987; Vol. 5, pp 634-774.
(38) Greenaway, F. T.; Pezeslik, A.; Cordes, A. W.; Noble, M. C.; Sorenson, J. R. J. Inorg. Chim. Acta 1984, 93, 67-71.
(39) Johnson, C. K., ORTEP II, Report ORNL-5138; Report ORNL-5138, Oak Ridge National Laboratories, Oak Ridge, Tennesee, 1976.
(40) Greenaway, F. T.; Riviere, E.; Girerd, J. J.; Labouze, X.; Morgant, G.; Viossat, B.; Daran, J. C.; Roch Arveiller, M.; Dung, N.-H. J. Inorg. Biochem. 1999, 76, 19-27.
(41) Maspoch, D.; Ruiz-Molina, D.; Wurst, K.; Vidal-Gancedo, J.; Rovira, C.; Veciana, J. Dalton Trans. 2004, 1073-1082.
(42) Abuhijleh, A. L.; Woods, C. J. Chem. Soc. Dalton Trans. 1992, 1249-1252.
(43) Valach, F.; Tokarcik, M.; Kubinec, P.; Melnik, M.; Macà\check{s}kovà, L. Polyhedron 1997, 16, 1461-1464.
(44) Abu Hijleh, A. L. Polyhedron 1989, 8, 2777-2783.
(45) Bhirud, R. G.; Srivastava, T. S. Inorg. Chim. Acta 1990, 173, 121-125.
(46) Hocking, R. K.; Hambley, T. W. Inorg. Chem. 2003, 42, 2833-2835.
(47) Blessing, R.H.; Acta Cryst. (1995) A51 33-38. Sheldrick, G.M.; SADABS. Empirical absorption correction program for area detector data. University of Gottingen, Germany, 1996.

1. A complex of formula (1):
[Cu(η²-L¹)₂L₂]^{p} (1)
wherein “η²-L¹” is a bidentate ligand of the formula L¹: 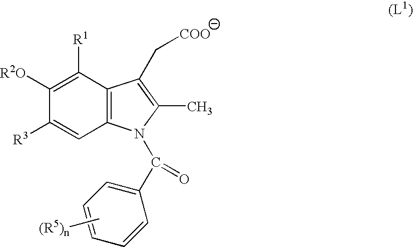
wherein:
R¹ is H or halo;
R² is H; a C₁ to C₆ alkyl, an alkenyl or an alkynyl, where the C₁ to C₆ alkyl, alkenyl or alkynyl may be optionally substituted; or 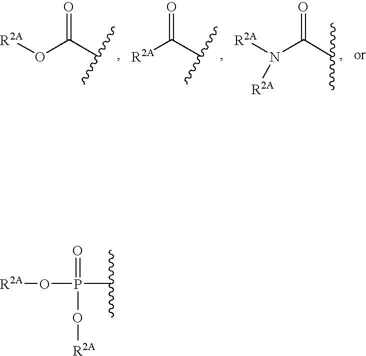
wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;
R³ is H or halo;
each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted;
n is 1, 2, 3, 4 or 5;
each L is independently selected and is a monodentate ligand;
and p is the charge of the complex.

2. A complex according to claim 1, wherein each R⁵ is a halo substituent.

3. A complex according to claim 2, wherein n is 1, 2 or 3 and each R⁵ is independently selected from Cl and Br.

4. A complex according to claim 1, wherein L¹ is the anion of indomethacin.

5. A complex according to claim 1, wherein L is a ligand containing an N-heterocyclic group.

6. A complex according to claim 1, wherein L is pyrrolidine or imidazole.

7. A pharmaceutical composition comprising a complex according to claim 1 and a pharmaceutically acceptable carrier.

8. A composition according to claim 7, wherein the composition is suitable for oral, rectal, nasal, topical, opthalmological, vaginal or parenteral administration.

9. A composition according to claim 8, wherein the composition is suitable for oral administration.

10. A method of treating an inflammatory condition in a human or animal, the method comprising administrating to the human or animal a therapeutically effective amount of a complex of formula (1):
[Cu(η²-L¹)₂L₂]^{p} (1)
wherein “η²-L¹” is a bidentate ligand of the formula L¹: 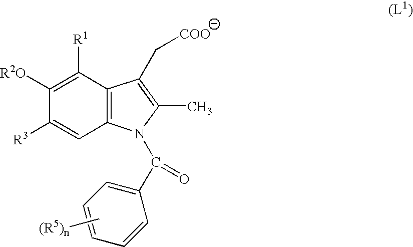
wherein:
R¹ is H or halo;
R² is H; a C₁ to C₆ alkyl, an alkenyl or an alkynyl, where the C₁ to C₆ alkyl, alkenyl or alkynyl may be optionally substituted; or 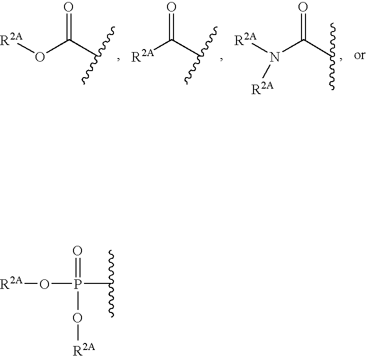
wherein each R^{2A} is independently selected from the group consisting of H, C₁ to C₆ alkyl, alkenyl, alkynyl, aryl, cycloalkyl and arylalkyl, where the C₁ to C₆ alkyl, alkenyl, alkynyl, aryl, cycloalkyl or arylalkyl may be optionally substituted;
R³ is H or halo;
each R⁵ is independently selected from the group consisting of halo, —CH₃, —CN, —OCH₃, —SCH₃ and —CH₂CH₃, where the —CH₃, —OCH₃, —SCH₃ or —CH₂CH₃ may be optionally substituted;
n is 1, 2, 3, 4 or 5;
each L is independently selected and is a monodentate ligand; and
p is the charge of the complex.

11. A method according to claim 10, wherein each R⁵ is a halo substituent.

12. A method according to claim 11, wherein n is 1, or 3 and each R⁵ is independently selected from Cl and Br.

13. A method according to claim 10, wherein L¹ is the anion of indomethacin.

14. A method according to claim 10, wherein L is a ligand containing an N-heterocyclic group.

15. A complex according to claim 10, wherein L is pyrrolidine or imidazole.

16. A method according to claim 10, wherein the animal is selected from the group consisting of a dog, a cat, a cow, a horse, human being, and a camel.

17. A method according to claim 10, wherein the complex is administered orally, rectally, by nasal spray, topically, opthalmologically, vaginally or parenterally.

18. A method according to claim 17, wherein the complex is administered orally.

19. (canceled)
